(19) United States
(12) Patent Application Publication
(10) Pub. No.: US 2022/0211346 A1
(43) Pub. Date: Jul. 7, 2022

(54) METHODS AND APPARATUSES FOR DISPLAYING ULTRASOUND DISPLAYS ON A FOLDABLE PROCESSING DEVICE

(71) Applicant: BFLY Operations, Inc., Guilford, CT (US)

(72) Inventors: David Elgena, Orlando, FL (US); Jason Gavris, New York, NY (US); Brian Shin, New York, NY (US); Karl Thiele, St. Petersburg, FL (US); Teresa Lopez, Plainville, CT (US)

(73) Assignee: BFLY Operations, Inc., Guilford, CT (US)

(21) Appl. No.: 17/566,538

(22) Filed: Dec. 30, 2021

Related U.S. Application Data
(60) Provisional application No. 63/133,774, filed on Jan. 4, 2021.

(51) Int. Cl. A61B 8/00; G09F 9/30; A61B 8/08

(52) U.S. Cl. CPC A61B 8/462; G09F 9/301; A61B 8/463; A61B 8/466; A61B 8/56; A61B 8/08; A61B 8/464; A61B 8/488; A61B 8/468; A61B 8/465

(57) ABSTRACT

A foldable processing device coupled to an ultrasound device is disclosed. In some embodiments, the foldable processing device may include a first panel having a first display screen, a second panel having a second display screen, and one or more hinges. The first panel and the second panel may be rotatably coupled by the one or more hinges. The foldable processing device may be in operative communication with an ultrasound device and configured to present different particular displays on the first and second display screens. In some embodiments, the foldable processing device may include a first panel, a second panel, a display screen, and one or more hinges. The first panel and the second panel may be rotatably coupled by the one or more hinges such that the display screen folds upon itself. The foldable processing device may be in operative communication with an ultrasound device and configured to present different particular displays on first and second portions of the display screen.

### CROSS-REFERENCE TO RELATED APPLICATIONS

The present application claims the benefit under 35 U.S.C. § 119(e) of U.S. Patent App. Ser. No. 63/133,774, filed Jan. 4, 2021 under Attorney Docket No. B1348.70194US00, and entitled “METHODS AND APPARATUSES FOR DISPLAYING ULTRASOUND DISPLAYS ON A FOLDABLE PROCESSING DEVICE,” which is hereby incorporated by reference herein in its entirety.

### FIELD

Generally, the aspects of the technology described herein relate to ultrasound displays. Certain aspects relate to displaying ultrasound displays on a foldable processing device.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to an aspect of the present technology, a foldable processing device is provided, wherein: the foldable processing device comprises a first panel comprising a first display screen, a second panel comprising a second display screen; and one or more hinges. The first panel and the second panel are rotatably coupled by the one or more hinges. The foldable processing device is in operative communication with an ultrasound device.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a top view of a foldable processing device in an open configuration, in accordance with certain embodiments described herein.

FIG. 2 illustrates another top view of the foldable processing device of FIG. 1 in the open configuration, in accordance with certain embodiments described herein.

FIG. 3 illustrates a side view of the foldable processing device of FIG. 1 in a folded configuration, in accordance with certain embodiments described herein.

FIGS. 4 and 5 illustrate the foldable processing device of FIG. 1 when operating in biplane imaging mode, in accordance with certain embodiments described herein.

FIGS. 6 and 7 illustrate the foldable processing device of FIG. 1 when operating in pulsed wave Doppler mode, in accordance with certain embodiments described herein.

FIGS. 8 and 9 illustrate the foldable processing device of FIG. 1 when operating in M-mode imaging, in accordance with certain embodiments described herein.

FIGS. 10 and 11 illustrate respective processes for using the foldable processing device of FIG. 1 to display ultrasound displays, in accordance with certain embodiments described herein.

FIG. 12 illustrates the foldable processing device of FIG. 1 when imaging the heart, in accordance with certain embodiments described herein.

FIGS. 13 and 14 illustrate respective processes for using the foldable processing device of FIG. 1 to display ultrasound displays, in accordance with certain embodiments described herein.

FIGS. 15 and 16 illustrate respective processes for using the foldable processing device of FIG. 1 to display ultrasound displays, in accordance with certain embodiments described herein.

FIG. 17 illustrates the foldable processing device of FIG. 1 when performing ultrasound imaging, in accordance with certain embodiments described herein.

FIG. 18 illustrates the foldable processing device of FIG. 1 when operating in a telemedicine mode, in accordance with certain embodiments described herein.

FIG. 19 illustrates the foldable processing device of FIG. 1 when retrieving a saved ultrasound image or images, in accordance with certain embodiments described herein.

FIG. 20 illustrates a process for using the foldable processing device of FIG. 1 to retrieve saved ultrasound image(s), in accordance with certain embodiments described herein.

FIG. 21 illustrates the foldable processing device of FIG. 1 when imaging the heart, in accordance with certain embodiments described herein.

FIG. 22 illustrates the foldable processing device of FIG. 1 when imaging the heart, in accordance with certain embodiments described herein.

FIG. 23 illustrates the foldable processing device of FIG. 1 when performing ultrasound imaging and documentation, in accordance with certain embodiments described herein.

FIG. 24 illustrates a process for using the foldable processing device of FIG. 1 to view ultrasound images in real-time and to freeze ultrasound images on a display screen, in accordance with certain embodiments described herein.

FIG. 25 illustrates a schematic block diagram of an example ultrasound system upon which various aspects of the technology described herein may be practiced.

FIG. 26 illustrates a top view of a foldable processing device in an open configuration, in accordance with certain embodiments described herein.

FIG. 27 illustrates another top view of the foldable processing device of FIG. 26 in the open configuration, in accordance with certain embodiments described herein.

FIG. 28 illustrates a side view of the foldable processing device of FIG. 26 in a folded configuration, in accordance with certain embodiments described herein.

FIG. 29 illustrates a schematic block diagram of an example ultrasound system upon which various aspects of the technology described herein may be practiced.

### DETAILED DESCRIPTION

Recently, foldable processing devices, which may be, for example, mobile smartphones or tablets, have become available. Some foldable devices include two different display screens. In an open configuration, the two display screens are both visible to a user. The foldable processing device can fold into a compact closed configuration, which may be helpful for portability and storage, for example. Some foldable devices include one foldable display screen that can fold along a hinge, which may allow for a relatively large display screen when the device is open while also allowing for a relatively small form factor when the device is folded. Such foldable devices may be considered to have two display screen portions, one on each side of the hinge.

The inventors have recognized that the two display screens or the two display screen portions of a foldable processing device may be helpful for ultrasound imaging. Recently, ultrasound devices that are in operative communication (e.g., over a wired or wireless communication link) with processing devices such as mobile smartphones and tablets have become available. Certain ultrasound imaging modes may include two different displays. For example, biplane imaging may include simultaneous display of two types of ultrasound images, one along an azimuthal plane and one along an elevational plane. In biplane imaging mode, a foldable processing device in operative communication with an ultrasound device may be configured to simultaneously display ultrasound images along the azimuthal plane on one display screen or one display screen portion and ultrasound images along the elevational plane on the other display screen or the other display screen portion. As another example, pulsed wave Doppler imaging may include simultaneous display of ultrasound images and a velocity trace. In pulsed wave Doppler imaging mode, a foldable processing device in operative communication with an ultrasound device may be configured to display ultrasound images on one display screen or one display screen portion and a velocity trace on the other display screen or other display screen portion. As another example, M-mode imaging may include simultaneous display of ultrasound images and an M-mode trace. In M-mode, a foldable processing device in operative communication with an ultrasound device may be configured to display ultrasound images on one display screen or one display screen portion and an M-mode trace on the other display screen or other display screen portion. Compared with displaying two ultrasound displays on one display screen, displaying two ultrasound displays each on a different display screen of a foldable processing device may be helpful in that the displays may be larger and easier for a user to see and manipulate. Similarly, compared with displaying two ultrasound displays on one display screen of a non-foldable device, displaying two ultrasound displays each on one portion of a single foldable display screen may be helpful in that the displays may be larger and easier for a user to see and manipulate.

Additionally, the inventors have recognized that the two display screens or two display screen portions of a foldable processing device may be used for other aspects of ultrasound imaging as well. For example, one display screen or display screen portion may display an ultrasound image while the other display screen or display screen portion may display ultrasound imaging actions, a quality indicator, ultrasound imaging controls, a telemedicine interface, saved ultrasound images, 2D and 3D ultrasound image visualizations, and/or fillable documentation.

Various aspects of the present disclosure may be used alone, in combination, or in a variety of arrangements not explicit in the embodiments described in the foregoing and is therefore not limited in its application to the details and arrangement of components set forth in the foregoing description or illustrated in the drawings. For example, aspects described in one embodiment may be combined in any manner with aspects described in other embodiments.

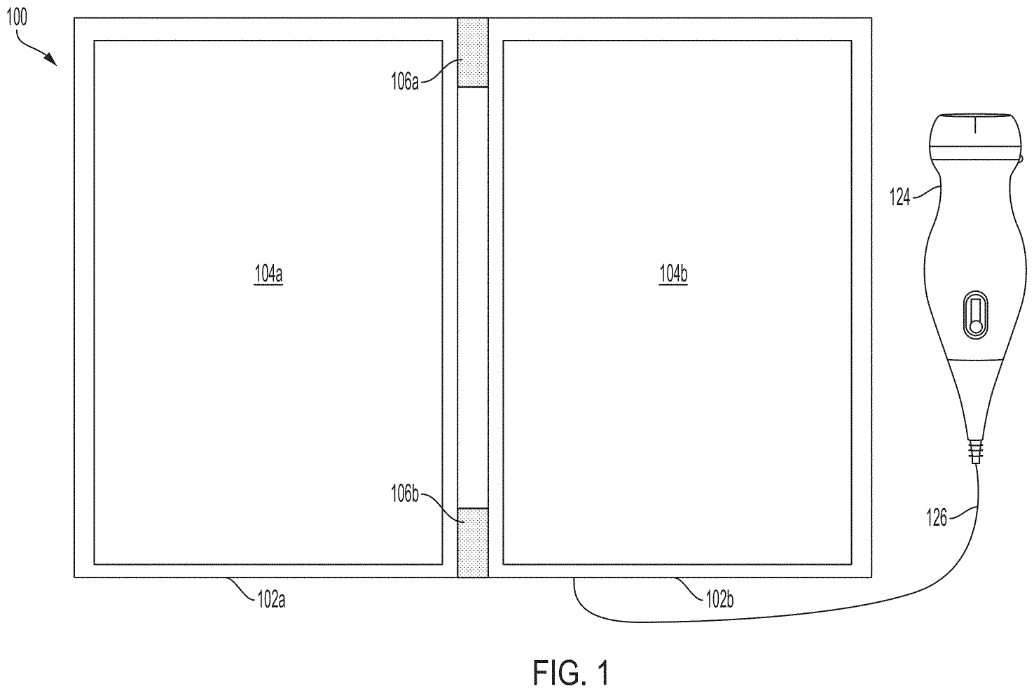

FIG. 1 illustrates a top view of a foldable processing device 100 in an open configuration, in accordance with certain embodiments described herein. The foldable processing device 100 may be any type of processing device, such as a mobile smartphone or a tablet. The foldable processing device 100 includes a first panel 102a, a second panel 102b, a first hinge 106a, and a second hinge 106b. The first panel 102a includes a first display screen 104a. The second panel 102b includes a second display screen 104b. The first panel 102a and the second panel 102b are rotatably coupled by the first hinge 104 and the second hinge 106. FIG. 1 further illustrates an ultrasound device 124 and a cable 126. The cable 126 extends between the ultrasound device 124 and the foldable processing device 100. The foldable processing device 100 may be in operative communication with the ultrasound device 124. Thus, the foldable processing device 100 may communicate with the ultrasound device 124 in order to control operation of the ultrasound device 124 and/or the ultrasound device 124 may communicate with the foldable processing device 100 in order to control operation of the foldable processing device 100. The cable 126 may be, for example, an Ethernet cable, a Universal Serial Bus (USB) cable, or a Lightning cable, or any other type of communications cable, and may facilitate communication between the foldable processing device 100 and the ultrasound device 124 over a wired communication link. In some embodiments, the cable 126 may be absent, and the foldable processing device 100 and the ultrasound device 124 may communicate over a wireless communication link (e.g., over a BLUETOOTH, WiFi, or ZIGBEE wireless communication link).

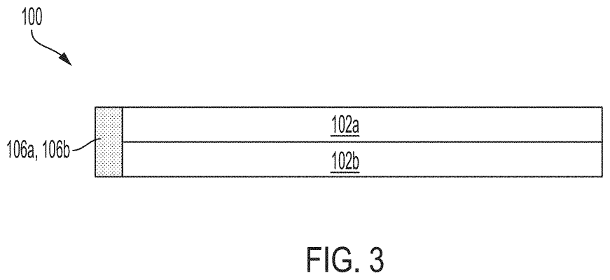

FIG. 1 displays an open configuration for the foldable processing device 100 in which the first panel 102a and the second panel 102b are substantially coplanar, and the first display screen 104a and the second display screen 104b are visible to a user. The first hinge 106a and the second hinge 106b enable the first panel 102a and/or the second panel 102b to rotate about the first hinge 106a and the second hinge 106b such that the foldable processing device 100 goes from the open configuration to a folded configuration, as illustrated in FIG. 3.

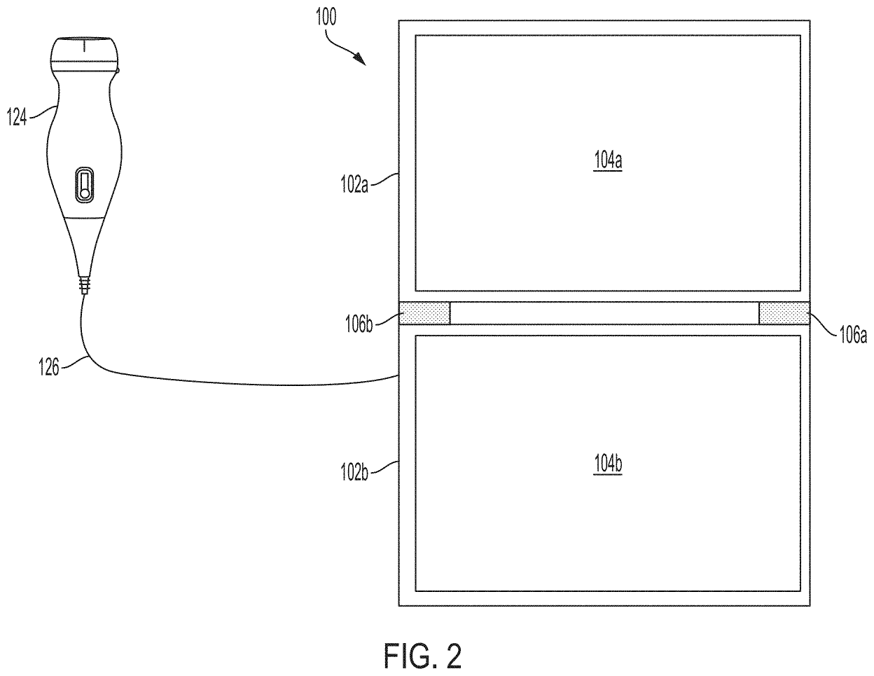

FIG. 2 illustrates another top view of the foldable processing device 100 in the open configuration, in accordance with certain embodiments described herein. The foldable processing device 100 is illustrated rotated from the orientation in FIG. 1. In some embodiments, in response to rotation of the foldable processing device 100 from the orientation in FIG. 1 to the orientation in FIG. 2, or vice versa, the foldable processing device 100 may cause the displays that are displayed on the first display screen 104a and/or the second display screen 104b to rotate as well. The configuration of FIG. 1 may be referred to as portrait mode while the configuration of FIG. 2 may be referred to as landscape mode.

FIG. 3 illustrates a side view of the foldable processing device 100 in a folded configuration, in accordance with certain embodiments described herein. In the folded configuration, the first display screen 104a and the second display screen 104b face each other, may be in contact with each other, and may not be visible to a user. The first panel 102a and the second panel 102b may be stacked one on top of another. The first hinge 106a and the second hinge 106b enable the first panel 102a and/or the second panel 102b to rotate about the first hinge 106a and the second hinge 106b such that the foldable processing device 100 goes from the folded configuration to the open configuration, as illustrated in FIGS. 1 and 2. The foldable processing device 100 may be more compact in the folded configuration than in the open configuration, while the open configuration may allow the first display screen 104a and the second display screen 104b to be visible.

While FIGS. 1-3 illustrate two hinges 106a and 106b, each at one end of the first panel 102a and the second panel 102b, some embodiments may have fewer or more hinges, and/or the hinge(s) may be at different locations. Additionally, other means for coupling the first panel 102a and the second panel 102b together such that the foldable processing device 100 can go from an open configuration to a foldable configuration may be used. For example, the foldable processing device may be formed of a foldable sheet of continuous material, such as a flexible circuit. It should also be appreciated that the size and shape of the foldable processing device 100, the first panel 102a, the second panel 102b, the first display screen 104a, and the second display screen 104b as illustrated is non-limiting, and that the foldable processing device 100, the first panel 102a, the second panel 102b, the first display screen 104a, and the second display screen 104b may have different sizes and/or shapes than illustrated.

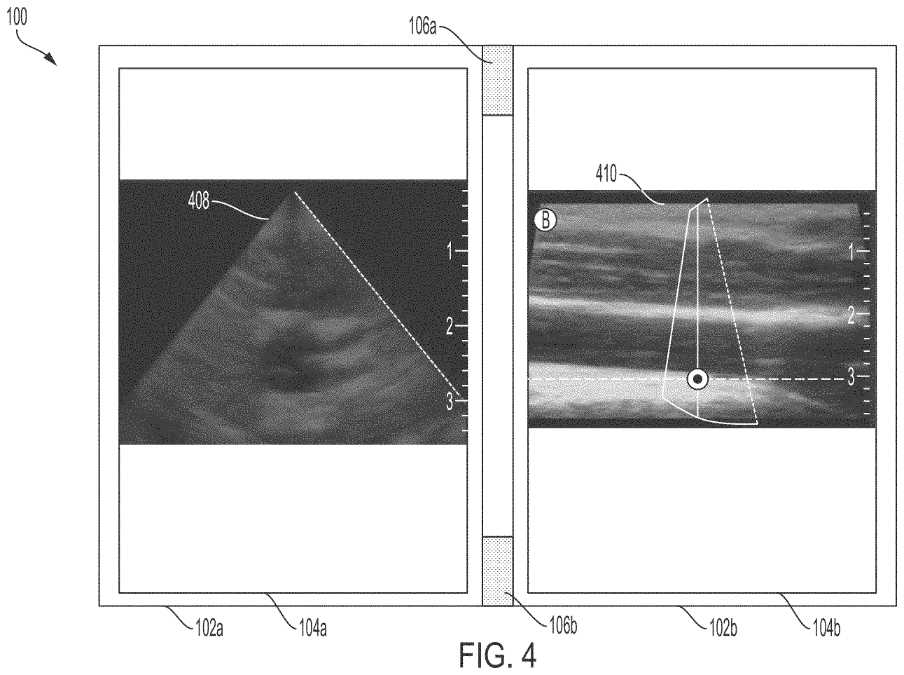

FIGS. 4-9 illustrate the foldable processing device 100 when operating in certain ultrasound imaging modes. Generally, the ultrasound imaging modes may include displaying at least two different displays. The foldable processing device 100 may be configured to display one of the displays related to the ultrasound imaging mode on the first display screen 104a and to display another of the displays related to the ultrasound imaging mode on the second display screen 104b. The foldable processing device 100 may display these two displays simultaneously. In some embodiments, the foldable processing device 100 may be configured to display these two displays related to the ultrasound imaging mode based on receiving a selection from a user (e.g., from a menu of options displayed on either or both of the first display screen 104a and the second display screen 104b) to operate in this ultrasound imaging mode. In some embodiments, the foldable processing device 100 may be configured to display these two displays related to the ultrasound imaging mode based on an automatic selection by the foldable processing device 100 (e.g., as part of an automatic workflow) to operate in this ultrasound imaging mode.

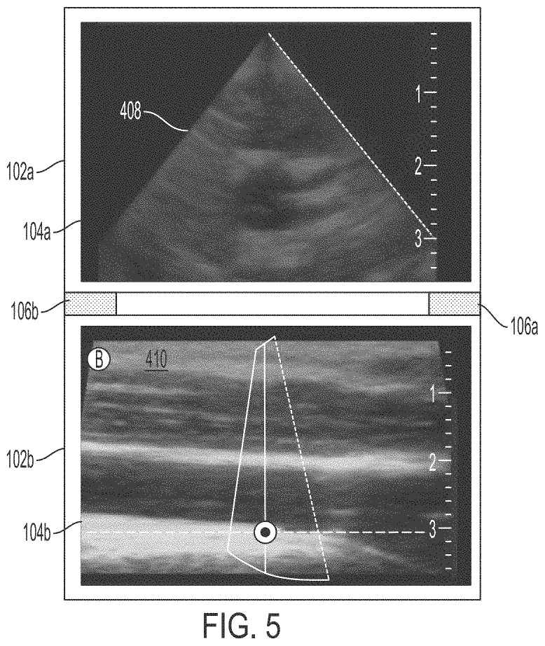

FIGS. 4 and 5 illustrate the foldable processing device 100 when operating in biplane imaging mode, in accordance with certain embodiments described herein. The first display screen 104a displays an ultrasound image along the elevational plane 408 and the second display screen 104b displays an ultrasound image along the azimuthal plane 410. The foldable processing device 100 may display the ultrasound image along the elevational plane 408 and the ultrasound image along the azimuthal plane 410 simultaneously.

The ultrasound device 124 with which the foldable processing device 100 is in operative communication, and specifically the ultrasound transducer array of the ultrasound device 124, may include an azimuthal dimension and an elevational dimension. The azimuthal dimension may be the dimension of the ultrasound transducer array that has more ultrasound transducers than the other dimension, which may be the elevational dimension. In some embodiments of biplane imaging mode, the foldable processing device 100 may configure the ultrasound device 124 to alternate collection of ultrasound images along the elevational plane 408 and collection of ultrasound images along the azimuthal plane 410. The ultrasound device 124 may collect the ultrasound images along the azimuthal plane 410 by transmitting and/or receiving ultrasound waves using an aperture (in other words, a subset of the ultrasound transducers) having a long dimension along the azimuthal dimension of the ultrasound transducer array of the ultrasound device 124. The ultrasound device 124 may collect the ultrasound images along the elevational plane 408 by transmitting and/or receiving ultrasound waves using an aperture having a long dimension along the elevational dimension of the ultrasound transducer array of the ultrasound device 124. Thus, alternating collection of the ultrasound images along the elevational plane 408 and collection of ultrasound images along the azimuthal plane 410 may include alternating collection of ultrasound images using one aperture and collection of ultrasound images using another aperture. In some embodiments, alternating collection of the ultrasound images along the elevational plane 408 and collection of the ultrasound images along the azimuthal plane 410 may include using the same aperture but with different beamforming parameters. Thus, alternating collection of the ultrasound images along the elevational plane 408 and collection of ultrasound images along the azimuthal plane 410 may include alternating generation of ultrasound images using one set of beamforming parameters and generation of ultrasound images using another set of beamforming parameters. The ultrasound device 124 may collect both types of ultrasound images without a user needing to rotate the ultrasound device 124.

In some embodiments, alternating collection of the ultrasound images may be at a rate in the range of approximately 15-30 Hz. In some embodiments, alternating collection of the ultrasound images may include collecting one ultrasound image along the elevational plane 408, then collecting one ultrasound image along the azimuthal plane 410, then collecting one ultrasound image along the elevational plane 408, etc. In some embodiments, alternating collection of the ultrasound images may include collecting one or more ultrasound images along the azimuthal plane 410, then collecting one or more ultrasound images along the elevational plane 408, then collecting one or more ultrasound images along the azimuthal plane 410, etc. In some embodiments, the foldable processing device 100 may be configured to receive each ultrasound image along the elevational plane 408 from the ultrasound device 124 and display it on the first display screen 104a (replacing the previously-displayed image on the first display screen 104a), and receive each ultrasound image along the azimuthal plane 410 from the ultrasound device 124 and display it on the second display screen 104b (replacing the previously-displayed image on the second display screen 104b). In some embodiments, the foldable processing device 100 may be configured to receive data for generating the ultrasound image along the elevational plane 408 from the ultrasound device 124, generate the ultrasound image along the elevational plane 408 from the data, and display it on the first display screen 104a (replacing the previously-displayed image on the first display screen 104a); the foldable processing device 100 may be configured to receive data for generating the ultrasound image along the azimuthal plane 410 from the ultrasound device 124, generate the ultrasound image along the azimuthal plane 410 from the data, and display it on the second display screen 104b (replacing the previously-displayed image on the second display screen 104b). In other words, the foldable processing device 100 may be configured to display a particular ultrasound image along the elevational plane 408 on the first display screen 104a until a new ultrasound image along the elevational plane 408 has been collected, and then display the newly collected ultrasound image along the elevational plane 408 instead of the previously collected ultrasound image along the elevational plane 408 on the first display screen 104a. The foldable processing device 100 may be configured to display a particular ultrasound image along the azimuthal plane 410 on the second display screen 104b until a new ultrasound image along the azimuthal plane 410 has been collected, and then display the newly collected ultrasound image along the azimuthal plane 410 instead of the previously collected ultrasound image along the azimuthal plane 410 on the second display screen 104b. In the example embodiments of FIG. 4, the ultrasound image along the elevational plane 408 and the ultrasound image along the azimuthal plane 410 contain certain orientation indicators, although certain embodiments may not include these orientation indicators. Further description of such orientation indicators and biplane imaging in general may be found in U.S. patent application Ser. No. 17/137,787 titled “METHODS AND APPARATUSES FOR MODIFYING THE LOCATION OF AN ULTRASOUND IMAGING PLANE,” filed on Dec. 30, 2020 and published as U.S. Pat. Pub. No. US 2021/0196237 A1 (and assigned to the assignee of the instant application), which is incorporated by reference herein in its entirety.

In some embodiments, the foldable processing device 100 may be configured to display the ultrasound image along the elevational plane 408 on the first display screen 104a and the ultrasound image along the azimuthal plane 410 on the second display screen 104b based on receiving a selection from a user (e.g., from a menu of options displayed on either or both of the first display screen 104a and the second display screen 104b) to operate in biplane imaging mode. In some embodiments, the foldable processing device 100 may be configured to display the ultrasound image along the elevational plane 408 on the first display screen 104a and the ultrasound image along the azimuthal plane 410 on the second display screen 104b based on an automatic selection by the foldable processing device 100 (e.g., as part of an automatic workflow) to operate in biplane imaging mode.

FIG. 4 illustrates the ultrasound image along the elevational plane 408 and the ultrasound image along the azimuthal plane 410 in portrait mode. FIG. 5 illustrates the ultrasound image along the elevational plane 408 and the ultrasound image along the azimuthal plane 410 in landscape mode. While the example embodiment of FIG. 4 illustrates the ultrasound image along the elevational plane 408 on the first display screen 104a and the ultrasound image along the azimuthal plane 410 on the second display screen 104b, in some embodiments the foldable processing device 100 may be configured to display the ultrasound image along the elevational plane 408 on the second display screen 104b and the ultrasound image along the azimuthal plane 410 on the first display screen 104a. While the example embodiment of FIG. 4 illustrates the ultrasound image along the elevational plane 408 on the left and the ultrasound image along the azimuthal plane 410 on the right, in some embodiments the foldable processing device 100 may be configured to display the ultrasound image along the elevational plane 408 on the right and the ultrasound image along the azimuthal plane 410 on the left. While the example embodiment of FIG. 5 illustrates the ultrasound image along the elevational plane 408 on the top and the ultrasound image along the azimuthal plane 410 on the bottom, in some embodiments the foldable processing device 100 may be configured to display the ultrasound image along the elevational plane 408 on the bottom and the ultrasound image along the azimuthal plane 410 on the top. It should also be appreciated that the foldable processing device 100 may display other items (e.g., control buttons and/or indicators) not illustrated in FIG. 4 or 5 on the first display screen 104a and/or the second display screen 104b.

Generally, in any of the figures herein, while the figure may illustrate an embodiment in which the foldable processing device 100 displays certain displays in portrait mode, in some embodiments the foldable processing device 100 may display the displays in landscape mode. While the figure may illustrate an embodiment in which the foldable processing device 100 displays certain displays in landscape mode, in some embodiments the foldable processing device 100 may display the displays in portrait mode. In any of the figures herein, while the figure may illustrate an embodiment in which a first display is on the first display screen 104a and a second display is on the second display screen 104b, in some embodiments the first display may be on the second display screen 104b and the second display may be on the first display screen 104a. In any of the figures herein, while the figure may illustrate an embodiment in which a first display is on the right and a second display is on the left, in some embodiments the first display may be on the left and the second display may be on the left. In any of the figures herein, while the figure may illustrate an embodiment in which a first display is on the top and a second display is on the bottom, in some embodiments the first display may be on the bottom and the second display may be on the top. In any of the figures herein, the foldable processing device 100 may display other items (e.g., control buttons and/or indicators) not illustrated in figure on the first display screen 104a and/or the second display screen 104b.

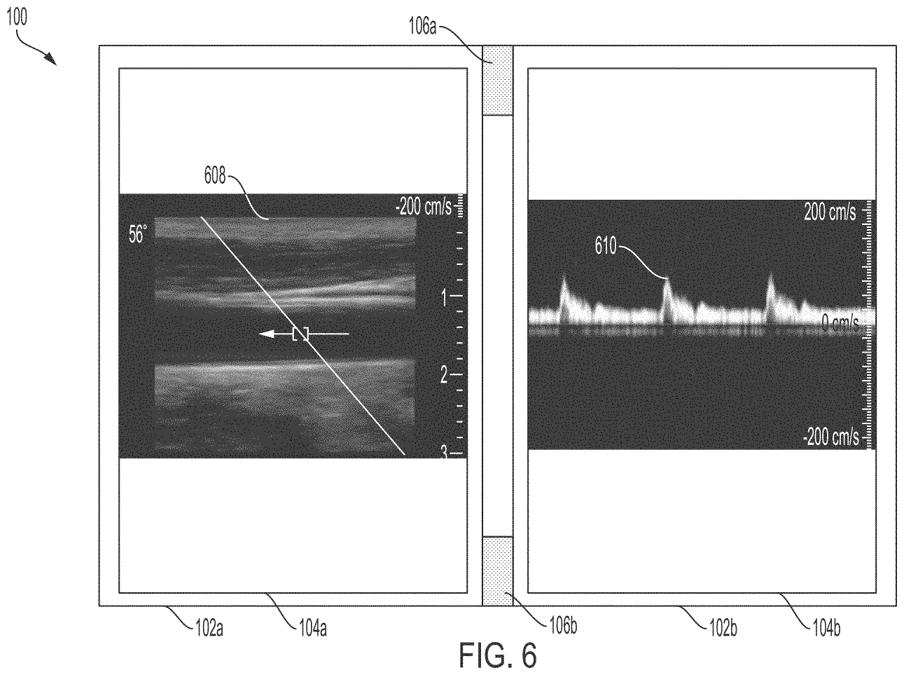

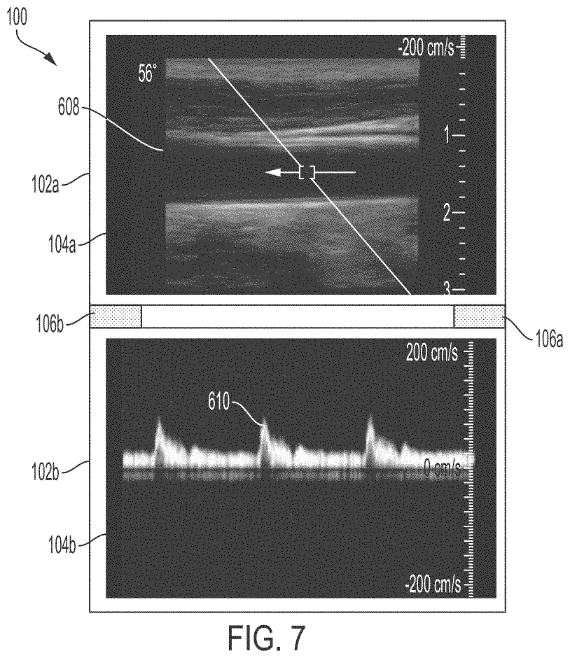

FIGS. 6 and 7 illustrate the foldable processing device 100 when operating in pulsed wave Doppler mode, in accordance with certain embodiments described herein. The first display screen 104a displays an ultrasound image 608 and the second display screen 104b displays a velocity trace 610. The foldable processing device 100 may display the ultrasound image 608 and the velocity trace 610 simultaneously.

In pulsed wave Doppler ultrasound imaging, ultrasound pulses may be directed at a particular portion of a subject in which something (e.g., blood) is flowing. This allows for measurement of the velocity of the flow. Generally, the parameters for pulse wave Doppler ultrasound imaging may include:

1. The portion of the subject where the flow velocity is to be measured, which may also be referred to as the sample volume;

2. The direction of the flow velocity to be measured. In other words, if flow occurs in an arbitrary direction, the component of the velocity of that flow along this particular selected direction may be the velocity measured; and

3. The direction in which the ultrasound pulses are transmitted from the ultrasound device 124, and in particular, from the transducer array of the ultrasound device 124, to the sample volume.

In the example embodiments of FIGS. 6 and 7, the above three parameters may be selected on the ultrasound image 608 that is displayed on the first display screen 104a, although it should be appreciated that in some embodiments, one or more of these parameters may be automatically selected by foldable processing device 100 based on the other selected parameters. Selection of these parameters may be accomplished using various controls and/or indicators superimposed on the ultrasound image 608 that is displayed on the first display screen 104a. The foldable processing device 100 may be configured to calculate the velocity through the selected sample direction and in the selected flow velocity direction for a particular ultrasound image 608. When another ultrasound image is collected, the foldable processing device 100 may display the newly collected ultrasound image 608 instead of the previously collected ultrasound image 608 on the first display screen 104a, and calculate the velocity for the newly collected ultrasound image 608. Thus, the foldable processing device 100 may calculate velocities as a function of time, and display the velocities as the velocity trace 610 on the second display screen 104b. Further description of selection of pulsed wave Doppler parameters and pulsed wave Doppler imaging in general may be found with reference to U.S. patent application Ser. No. 17/103,059 titled “METHODS AND APPARATUSES FOR PULSED WAVE DOPPLER ULTRASOUND IMAGING,” filed on Nov. 24, 2020 and published as U.S. Pat. Pub. No. US 2021/0153846 A1 (and assigned to the assignee of the instant application), which is incorporated by reference herein in its entirety.

In some embodiments, the foldable processing device 100 may be configured to display the ultrasound image 608 on the first display screen 104a and the velocity trace 610 on the second display screen 104b based on receiving a selection from a user (e.g., from a menu of options displayed on either or both of the first display screen 104a and the second display screen 104b) to operate in pulsed wave Doppler imaging mode. In some embodiments, the foldable processing device 100 may be configured to display the ultrasound image 608 on the first display screen 104a and the velocity trace 610 on the second display screen 104b based on an automatic selection by the foldable processing device 100 (e.g., as part of an automatic workflow) to operate in pulsed wave Doppler imaging mode.

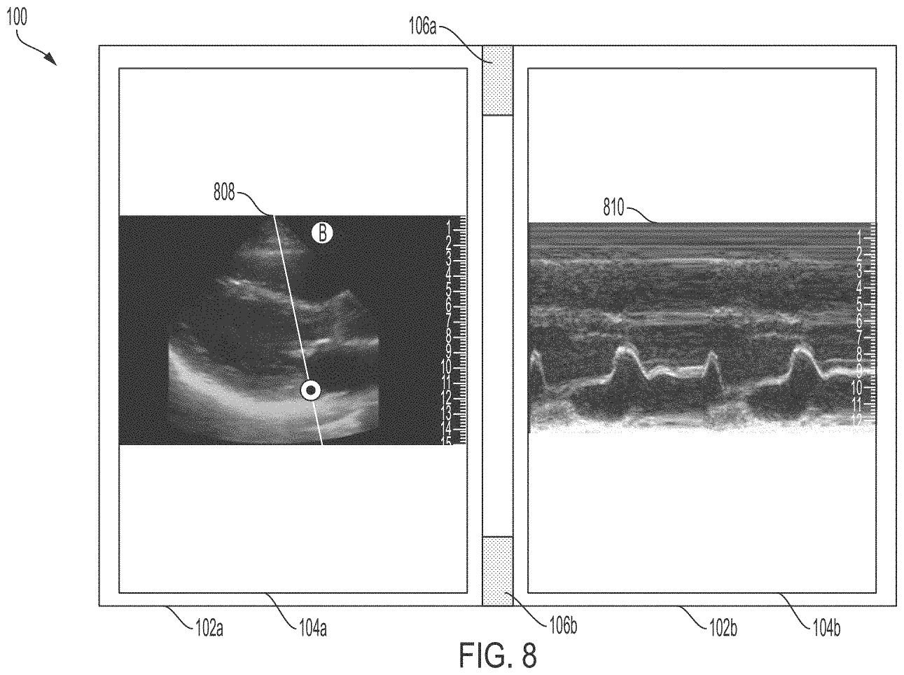

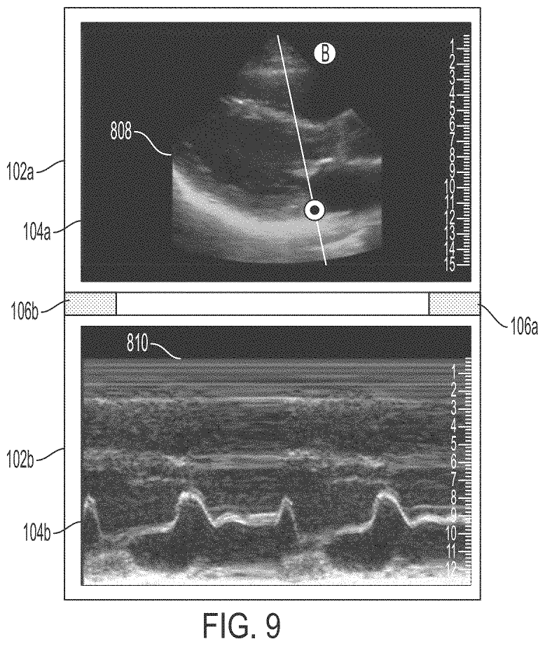

FIGS. 8 and 9 illustrate the foldable processing device 100 when operating in M-mode imaging, in accordance with certain embodiments described herein. The first display screen 104a displays an ultrasound image 808 and the second display screen 104b displays an M-mode trace 810. The foldable processing device 100 may display the ultrasound image 808 and the M-mode trace 810 simultaneously.

In M-mode, a user may select a line through an ultrasound image 808. As each successive ultrasound image 808 is collected, the foldable processing device 100 may determine the portion of the ultrasound image 808 that is along the line and add it adjacent to the portion of the previous ultrasound image 808 that is along that line to form the M-mode trace 810, which the foldable processing device 100 may display on the second display screen 104b. In the example embodiments of FIGS. 8 and 9, the line through the ultrasound image 808 is selected on an ultrasound image 808 that is displayed on the first display screen 104a. Selection of this parameter may be accomplished using various controls and/or indicators superimposed on the ultrasound image 808 that is displayed on the first display screen 104a.

In some embodiments, the foldable processing device 100 may be configured to display the ultrasound image 808 on the first display screen 104a and the M-mode trace 810 on the second display screen 104b based on receiving a selection from a user (e.g., from a menu of options displayed on either or both of the first display screen 104a and the second display screen 104b) to operate in M-mode. In some embodiments, the foldable processing device 100 may be configured to display the ultrasound image 808 on the first display screen 104a and the M-mode trace 810 on the second display screen 104b based on an automatic selection by the foldable processing device 100 (e.g., as part of an automatic workflow) to operate in M-mode.

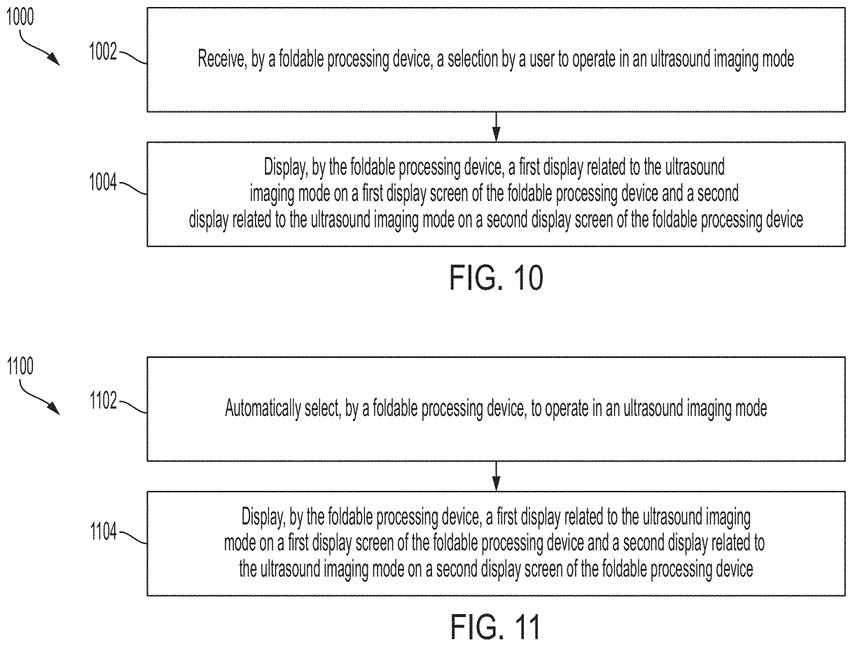

FIGS. 10 and 11 illustrate processes 1000 and 1100, respectively, for using the foldable processing device 100 to display ultrasound displays, in accordance with certain embodiments described herein. The process 1000 begins at act 1002. In act 1002, the foldable processing device 100 receives a selection by a user to operate in an ultrasound imaging mode. In some embodiments, the foldable processing device 100 may receive the selection by the user from a menu of options displayed on either or both of the first display screen 104a and the second display screen 104b. The ultrasound imaging mode may be, for example, biplane imaging mode, pulsed wave Doppler imaging mode, or M-mode imaging. The process 1000 proceeds from act 1002 to act 1004.

In act 1004, the foldable processing device 100 displays a first display related to the ultrasound imaging mode on the first display screen 104a of the foldable processing device 100 and a second display 104b related to the ultrasound imaging mode on the second display screen 104b of the foldable processing device 100. For example, if the ultrasound imaging mode is biplane imaging mode, the first display may be an ultrasound image along the elevational plane (e.g., the ultrasound image along the elevational plane 408) and the second display may be an ultrasound image along the azimuthal plane (e.g., the ultrasound image along the azimuthal plane 410). Further description of biplane imaging mode may be found with reference to FIGS. 4 and 5. As another example, if the ultrasound imaging mode is pulsed wave Doppler imaging mode, the first display may be an ultrasound image (e.g., the ultrasound image 608) and the second display may be a velocity trace (e.g., the velocity trace 610). Further description of pulsed wave Doppler imaging mode may be found with reference to FIGS. 6 and 7. As another example, if the ultrasound imaging mode is M-mode imaging, the first display may be an ultrasound image (e.g., the ultrasound image 808) and the second display may be an M-mode trace (e.g., the M-mode trace 810). Further description of M-mode imaging may be found with reference to FIGS. 8 and 9.

The process 1100 begins at act 1102. In act 1102, the foldable processing device 100 automatically selects to operate in an ultrasound imaging mode. In some embodiments, the foldable processing device 100 may automatically select to operate in the ultrasound imaging mode as part of an automatic workflow. The ultrasound imaging mode may be, for example, biplane imaging mode, pulsed wave Doppler imaging mode, or M-mode imaging. The process 1100 proceeds from act 1102 to act 1104. Act 1104 is the same as act 1004.

While the above description has focused on biplane imaging mode, pulsed wave Doppler imaging mode, and M-mode image, these are non-limiting. In any ultrasound imaging mode that includes display of more than one display, the foldable processing device 100 may display one of the displays on the first display screen 104a and another display on the second display screen 104b.

The foldable processing device 100 may be configured to display an ultrasound image on the first display screen 104a and to display ultrasound imaging actions related to the anatomical portion being imaged on the second display screen 104b (or vice versa). The anatomical portion may be, for example, an anatomical region, structure, or feature. The foldable processing device 100 may display the ultrasound image and the ultrasound imaging actions simultaneously. In some embodiments, the foldable processing device 100 may be configured to display the ultrasound image and the ultrasound imaging actions related to the anatomical portion based on receiving a selection from a user (e.g., from a menu of options displayed on either or both of the first display screen 104a and the second display screen 104b) to image the anatomical portion. In some embodiments, the foldable processing device 100 may be configured to display the ultrasound image and the ultrasound imaging actions related to the anatomical portion based on an automatic selection by the foldable processing device 100 (e.g., as part of an automatic workflow) to image the anatomical portion.

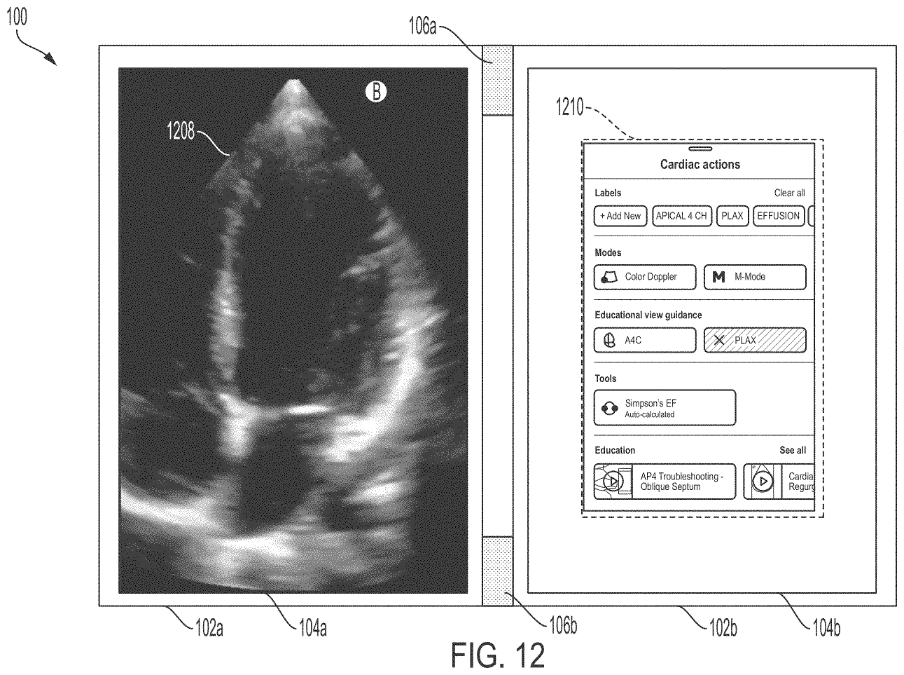

FIG. 12 illustrates the foldable processing device 100 when imaging the heart, in accordance with certain embodiments described herein. The first display screen 104a displays an ultrasound image 1208 and the second display screen 104b displays actions related to ultrasound imaging of the heart 1210. The ultrasound image 1208 may be the most recently displayed ultrasound image, and may be frozen on the display screen 104a or updated in real time as subsequent ultrasound images are collected. The actions related to ultrasound imaging of the heart 1210 include actions that, when selected by the user from the second display screen 104b, cause the foldable processing device 100 to perform actions related to ultrasound imaging of the heart 1210. As illustrated, such actions may include enabling a user to annotate the ultrasound image 1208 with annotations specific to the heart, to be guided by the foldable processing device 100 to collect an ultrasound image of the heart, to cause the foldable processing device 100 to automatically perform a calculation related to the heart (e.g., calculating ejection fraction), and to view videos related to ultrasound imaging of the heart. It should be appreciated that the actions related to ultrasound imaging of the heart 1210 described above are non-limiting, and other actions may be included, or certain actions may be absent. The foldable processing device 100 may display the ultrasound image 1208 and the actions related to ultrasound imaging of the heart 1210 simultaneously.

In some embodiments, the foldable processing device 100 may be configured to display the ultrasound image 1208 on the first display screen 104a and the actions related to ultrasound imaging of the heart 1210 on the second display screen 104b based on receiving a selection from a user (e.g., from a menu of options displayed on either or both of the first display screen 104a and the second display screen 104b) to image the heart. Such selection may cause the foldable processing device 100 to configure the ultrasound device 124 with predetermined imaging parameters (which may be referred to as a preset) optimized for imaging the heart. In some embodiments, the foldable processing device 100 may be configured to display the ultrasound image 1208 on the first display screen 104a and the actions related to ultrasound imaging of the heart 1210 on the second display screen 104b based on an automatic selection by the foldable processing device 100 (e.g., as part of an automatic workflow) to image the heart.

While the above description has focused on actions related to ultrasound imaging of the heart, it should be appreciated that this application is not limited to the heart, and foldable processing device 100 may display actions related to ultrasound imaging of other anatomical portions. For example, for imaging the lungs, the foldable processing device 100 may display actions for enabling a user to annotate an ultrasound image with annotations specific to the lungs, to be guided by the foldable processing device 100 to collect an ultrasound image of the lungs, to cause the foldable processing device 100 to automatically perform a calculation related to the lungs (e.g., counting B-lines), and to view videos related to ultrasound imaging of the lungs. As another example, for imaging the bladder, the foldable processing device 100 may display actions for enabling a user to annotate an ultrasound image with annotations specific to the bladder, to be guided by the foldable processing device 100 to collect an ultrasound image of the bladder, to cause the foldable processing device 100 to automatically perform a calculation related to the bladder (e.g., calculating bladder volume), and to view videos related to ultrasound imaging of the bladder.

As another example, for obstetric imaging, the foldable processing device 100 may display actions for enabling a user to annotate an ultrasound image with annotations specific to obstetrics, to be guided by the foldable processing device 100 to collect an ultrasound image of a fetus, to cause the foldable processing device 100 to automatically perform a calculation related to obstetrics (e.g., calculating gestational age, estimated delivery date, fetal weight, or amniotic fluid index), and to view videos related to ultrasound imaging of fetuses.

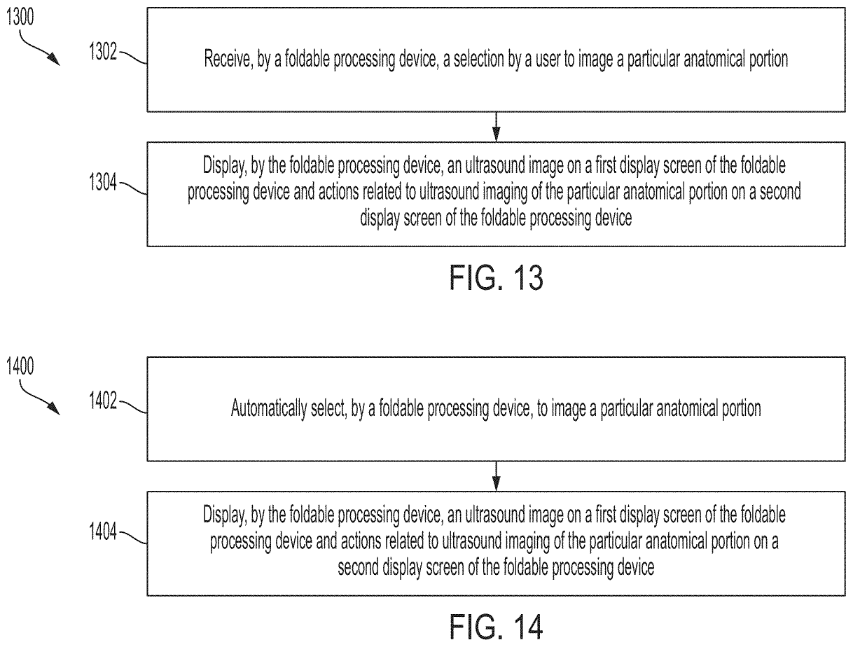

FIGS. 13 and 14 illustrate processes 1300 and 1400, respectively, for using a foldable processing device 100 to display ultrasound displays, in accordance with certain embodiments described herein. The process 1300 begins at act 1302. In act 1302, the foldable processing device 100 receives a selection by a user to image a particular anatomical portion (e.g., an anatomical region, structure, or feature). Such selection may cause the foldable processing device 100 to configure the ultrasound device 124 with predetermined imaging parameters (which may be referred to as a preset) optimized for imaging the anatomical portion. In some embodiments, the foldable processing device 100 may receive the selection by the user from a menu of options displayed on either or both of the first display screen 104a and the second display screen 104b. The process 1300 proceeds from act 1302 to act 1304.

In act 1304, the foldable processing device 100 displays an ultrasound image (e.g., the ultrasound image 1208) on the first display screen 104a of the foldable processing device 100 and actions related to ultrasound imaging of the particular anatomical portion (e.g., the actions related to ultrasound imaging of the heart 1210) on the second display screen 104b of the foldable processing device 100. For example, the actions may include (but are not limited to) actions performed by the foldable processing device 100 that enable a user to annotate an ultrasound image with annotations specific to the particular anatomical portion, to be guided by the foldable processing device 100 to collect an ultrasound image of the particular anatomical portion, to cause the foldable processing device 100 to automatically perform a calculation related to the particular anatomical portion (e.g., calculation of ejection fraction for ultrasound imaging of the heart, counting of B-lines for ultrasound imaging of the lungs, calculation of bladder volume for ultrasound imaging of the bladder, or calculation of gestational age, estimated delivery date, fetal weight, or amniotic fluid index for obstetric imaging), and to view videos related to ultrasound imaging of the particular anatomical portion.

The process 1400 begins at act 1402. In act 1402, the foldable processing device 100 automatically selects to image a particular anatomical portion (e.g., an anatomical region, structure, or feature). Such selection may cause the foldable processing device 100 to configure the ultrasound device 124 with predetermined imaging parameters (which may be referred to as a preset) optimized for imaging the anatomical region. In some embodiments, the foldable processing device 100 may automatically select to image the particular anatomical portion as part of an automatic workflow. The process 1400 proceeds from act 1402 to act 1404. Act 1404 is the same as act 1304.

The foldable processing device 100 may be configured to display an ultrasound image on the first display screen 104a and to display an ultrasound image quality indicator related to the anatomical portion being imaged on the second display screen 104b (or vice versa). The anatomical portion may be, for example, an anatomical region, structure, or feature. The foldable processing device 100 may display the ultrasound image and the ultrasound image quality indicator simultaneously. In some embodiments, the foldable processing device 100 may be configured to display the ultrasound image and the ultrasound image quality indicator related to the anatomical portion based on receiving a selection from a user (e.g., from a menu of options displayed on either or both of the first display screen 104a and the second display screen 104b) to image the anatomical portion. In some embodiments, the foldable processing device 100 may be configured to display the ultrasound image and the ultrasound imaging actions related to the anatomical portion based on an automatic selection by the foldable processing device 100 (e.g., as part of an automatic workflow) image the anatomical portion.

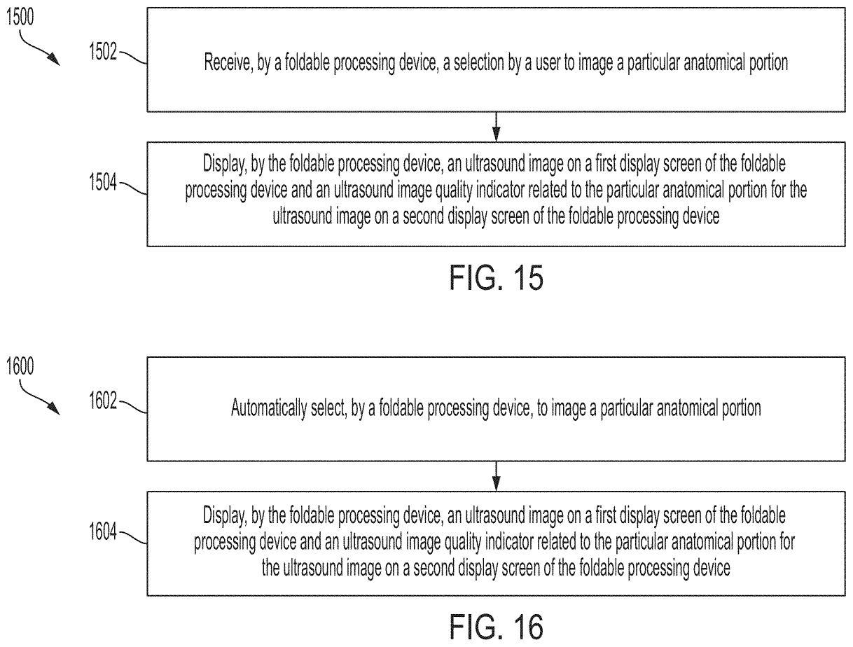

FIGS. 15 and 16 illustrate processes 1500 and 1600, respectively, for using a foldable processing device 100 to display ultrasound displays, in accordance with certain embodiments described herein. The process 1500 begins at act 1502, which is the same as act 1502. The process 1500 proceeds from act 1502 to act 1504. In act 1504, the foldable processing device 100 displays an ultrasound image (e.g., the ultrasound image 2208) on the first display screen 104a of the foldable processing device 100 and a quality indicator (e.g., the quality indicator 2212) related to the particular anatomical portion for the ultrasound image on the second display screen 104b of the foldable processing device 100. In some embodiments, the quality of the ultrasound image as indicated by the quality indicator may be based, at least in part, on a prediction of what proportion of experts (e.g., experts in the field of medicine, experts in a particular field of medicine, experts in ultrasound imaging, etc.) would consider the ultrasound image clinically usable as an ultrasound image of the particular anatomical region. In some embodiments, to determine the quality as indicated by the quality indicator, the foldable processing device 100 may use a statistical model trained to output such a prediction based on inputted ultrasound images. The quality indicator may be specific to ultrasound imaging of the particular anatomical portion in that it may indicate a low quality for ultrasound images of other anatomical portions despite such ultrasound images being high quality otherwise. This may be due to the statistical model being specifically trained to recognize ultrasound images of the particular anatomical region as high quality. The quality indicator may specifically indicate high qualities for ultrasound images predicted to be usable for certain purposes related to ultrasound imaging of the particular anatomical portion (e.g., calculation of ejection fraction for ultrasound imaging of the heart, counting of B-lines for ultrasound imaging of the lungs, or calculation of bladder volume for ultrasound imaging of the bladder). The quality indicator may indicate the quality textually, graphically, or both.

The process 1600 begins at act 1602, which is the same as act 1402. The process 1600 proceeds from act 1602 to act 1604, which is the same as act 1504.

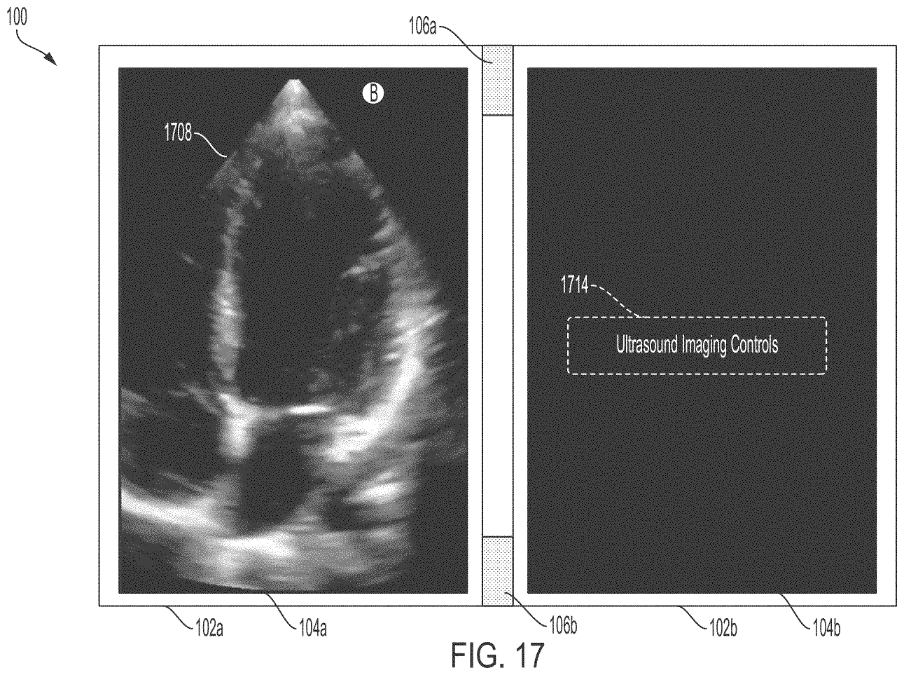

FIG. 17 illustrates the foldable processing device 100 when performing ultrasound imaging, in accordance with certain embodiments described herein. The first display screen 104a displays an ultrasound image 1708 and the second display screen 104b displays ultrasound imaging controls 1714. The ultrasound image 1708 may be the most recently displayed ultrasound image, and may be frozen on the display screen 104a or updated in real time as subsequent ultrasound images are collected. FIG. 17 generally indicates ultrasound imaging controls 1714, which may be used for ultrasound imaging for imaging of any anatomical portion and/or in any ultrasound imaging mode, but does not illustrate any specific ultrasound imaging controls. It should be appreciated that such ultrasound imaging controls may include, but are not limited to, controls for freezing the ultrasound image 1708, capturing the ultrasound image 1708 as a still image, recording ultrasound clips, adjusting gain, adjusting depth, adjusting time gain compensation (TGC), selecting the anatomical portion to be imaged (which may include selecting predetermined ultrasound imaging parameters optimized for imaging the anatomical portion, which may be referred to as a preset), selecting the ultrasound imaging mode, adding annotations to the ultrasound image 1708, and/or performing measurements on the ultrasound image 1708 (e.g., linear measurements or area measurements). It should be appreciated that the ultrasound imaging controls 1714 may include any of the controls described above, or other ultrasound imaging controls not specifically described.

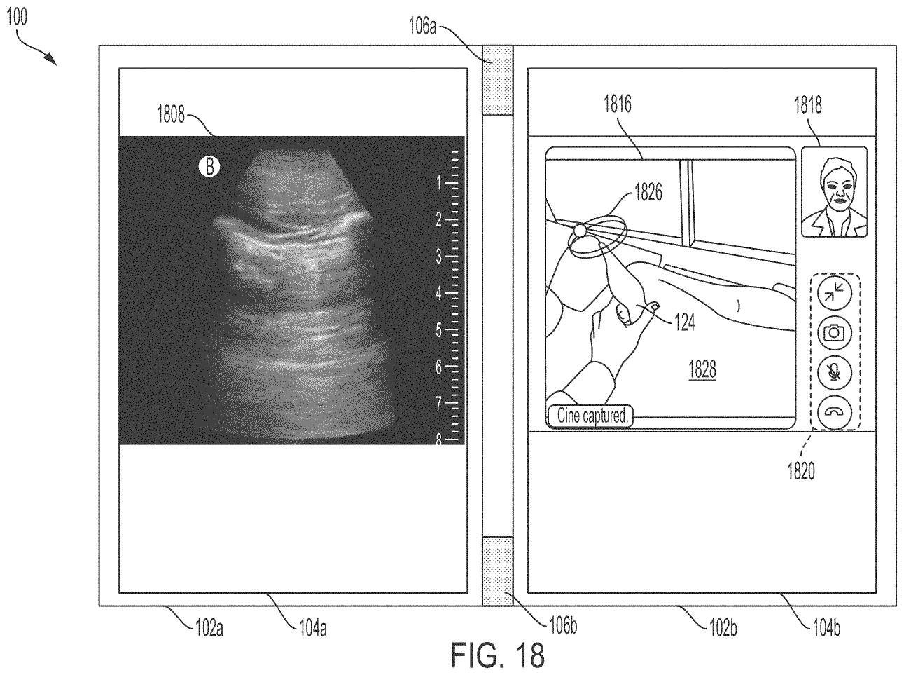

FIG. 18 illustrates the foldable processing device 100 when operating in a telemedicine mode, in accordance with certain embodiments described herein. Telemedicine may include a real-time call between a user (who is using the foldable processing device 100 and the ultrasound device 124) and a remote guide, in which the remote guide may help the user to use the ultrasound device 124 capture an ultrasound image from a subject 1828. The first display screen 104a displays an ultrasound image 1808 and the second display screen 104b displays a subject image 1816, a remote guide image 1818, and telemedicine controls 1820. The ultrasound image 1808 may be the most recently displayed ultrasound image, and may be frozen on the display screen 104a or updated in real time as subsequent ultrasound images are collected. The subject image 1816, the remote guide image 1818, and the telemedicine controls 1820 may together be considered a telemedicine interface, or a portion thereof. The subject image 1816 shows the subject 1828 being imaged, the ultrasound device 124, and an instruction 1826 for moving the ultrasound device 124 (although in some embodiments, one or more of these may be absent). The subject image 1816 may be a frame of a video captured by a camera of the foldable processing device 100. The ultrasound image 1808 may have been captured by the ultrasound device 124 shown in the subject image 1816 and from the subject 1828 shown in the subject image 1816. The remote guide image 1818 may be an image of the remote guide. The remote guide may transmit to the foldable processing device the instruction 1826 that is shown in the subject image 1816 to guide the user to capture an ultrasound image. The instruction 1826 may be, for example, an instruction to translate, rotate, or tilt the ultrasound device 124. The telemedicine controls 1820 include controls for changing the size of the subject image 1816, changing the orientation of the subject image 1816, muting a microphone on the foldable processing device 100, and ending the call with the remote guide, but in some embodiments, more or fewer of these controls may be present. Additionally, in some embodiments, one or more of the subject image 1816, the remote guide image 1818, and the telemedicine controls 1820 may be absent. Further description of telemedicine may be found in U.S. patent application Ser. No. 16/285,573, published as U.S. Patent Publication No. 2019/0261957 A1 and titled “METHODS AND APPARATUSES FOR TELE-MEDICINE,” filed on Feb. 26, 2019 (and assigned to the assignee of the instant application), which is incorporated by reference herein in its entirety; and U.S. patent application Ser. No. 16/735,019, published as U.S. Patent Publication No. 2020/0214682 A1 and titled “METHODS AND APPARATUSES FOR TELE-MEDICINE,” filed on Jan. 6, 2020 (and assigned to the assignee of the instant application), which is incorporated by reference herein in its entirety.

While FIG. 18 illustrates the ultrasound image 1808 on the first display screen 104a, in some embodiments the ultrasound image 1808 may be on the second display screen 104b. While FIG. 18 illustrates the subject image 1816 on the second display screen 104b, in some embodiments the subject image 1816 may be on the first display screen 104a. While FIG. 18 illustrates the remote guide image 1818 on the second display screen 104b, in some embodiments the remote guide image 1818 may be on the first display screen 104a. While FIG. 18 illustrates the telemedicine controls 1820 on the second display screen 104b, in some embodiments the telemedicine controls 1820 may be on the first display screen 104a.

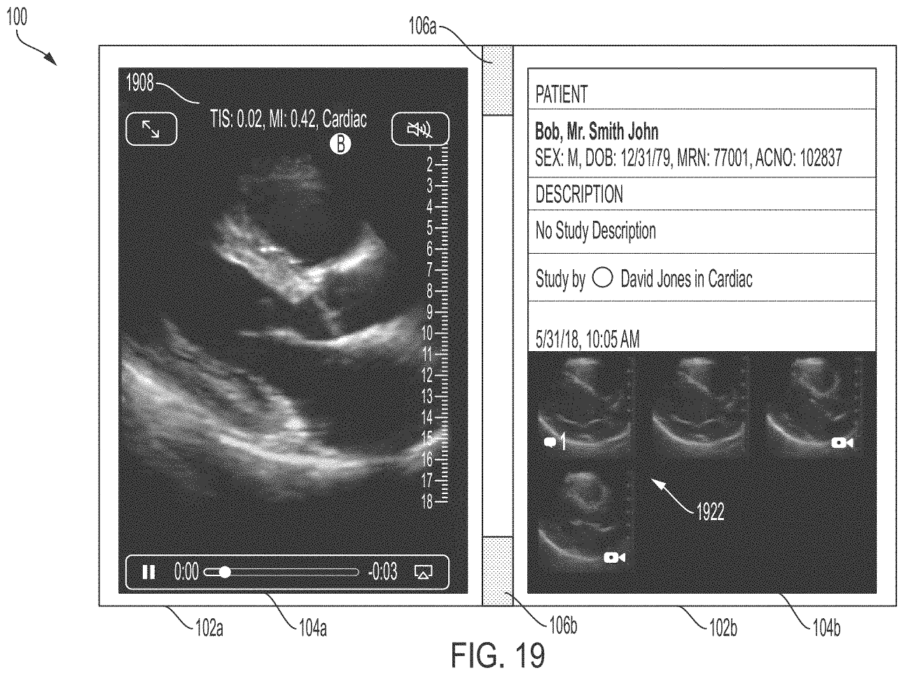

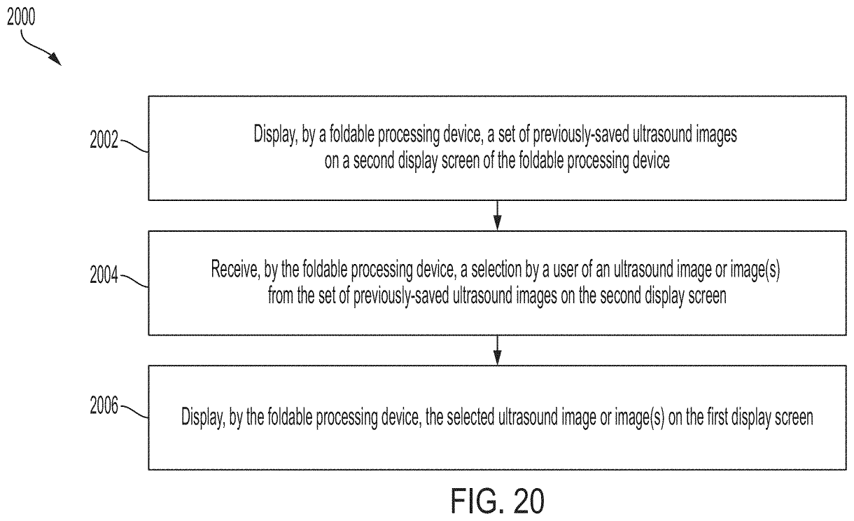

FIG. 19 illustrates the foldable processing device 100 when retrieving a saved ultrasound image or images, in accordance with certain embodiments described herein. The first display screen 104a displays an ultrasound image or images 1908 and the second display screen 104b displays a set of saved ultrasound images 1922. Each element of the set may be one ultrasound image or a clip of multiple ultrasound images. The set of saved ultrasound images 1922 includes the ultrasound image(s) 1908. In FIG. 19, each ultrasound image or clip of ultrasound images is displayed as a thumbnail, although in some embodiments they may be displayed in other manners, such as a list of titles of ultrasound images or clips. A user of the ultrasound device 124 may have captured multiple ultrasound images or clips and saved them to memory (e.g., on the foldable processing device 100 or on an external server), and these ultrasound images may be displayed as the set of saved ultrasound images 1922 for subsequent retrieval by the user and display on the first display screen 104a of the foldable processing device 100. Thus, upon receiving a selection from the user of one of the ultrasound images or one of the clips from the set of saved ultrasound images 1922 from the second display screen 104b (e.g., by the user touching or clicking on one of the thumbnails), the foldable processing device 100 may display the selected ultrasound image(s) 1908 on the first display screen 104a, as illustrated in FIG. 20. The display of the selected ultrasound image(s) 1908 on the first display screen 104a may be at a larger size than the size at which the selected ultrasound image(s) 1908 were displayed in the set of saved ultrasound images 1922 on the second display screen 104b (e.g., larger than a thumbnail). If the selected ultrasound image(s) 1908 are in the form of a clip, the foldable processing device 100 may play the clip.

FIG. 20 illustrates a process 2000 for using a foldable processing device 100 to retrieve saved ultrasound image(s), in accordance with certain embodiments described herein.

The process 2000 begins at act 2002. In act 2002, the foldable processing device 100 displays a set of saved ultrasound images (e.g., the saved ultrasound images 1922) on the second display screen 104b of the foldable processing device 100. Each element of the set may be one ultrasound image or a clip of multiple ultrasound images. Each ultrasound image or clip of ultrasound images in the set may be displayed, for example, as a thumbnail, or as a title in a list. A user of the ultrasound device 124 may have captured multiple ultrasound images or clips and saved them to memory (e.g., on the foldable processing device 100 or on an external server), and these ultrasound images may be displayed as the set of saved ultrasound images for subsequent retrieval by the user and display on the first display screen 104a of the foldable processing device 100. The process 2000 proceeds from act 2002 to act 2004.

In act 2004, the foldable processing device 100 receives a selection by a user of an ultrasound image or image(s) from the set of saved ultrasound images on the second display screen. For example, if the set is displayed as thumbnails, then the user may touch or click on one of the thumbnails. The process 2000 proceeds from act 2004 to act 2006.

In act 2006, the foldable processing device 100 displays the selected ultrasound image or image(s) (i.e., selected in act 2004) on the first display screen 104a. The display of the selected ultrasound image(s) on the first display screen 104a may be at a larger size than the size at which the selected ultrasound image(s) were displayed in the set of saved ultrasound images on the second display screen 104b (e.g., larger than a thumbnail). If the selected ultrasound image(s) are in the form of a clip, the foldable processing device 100 may play the clip.

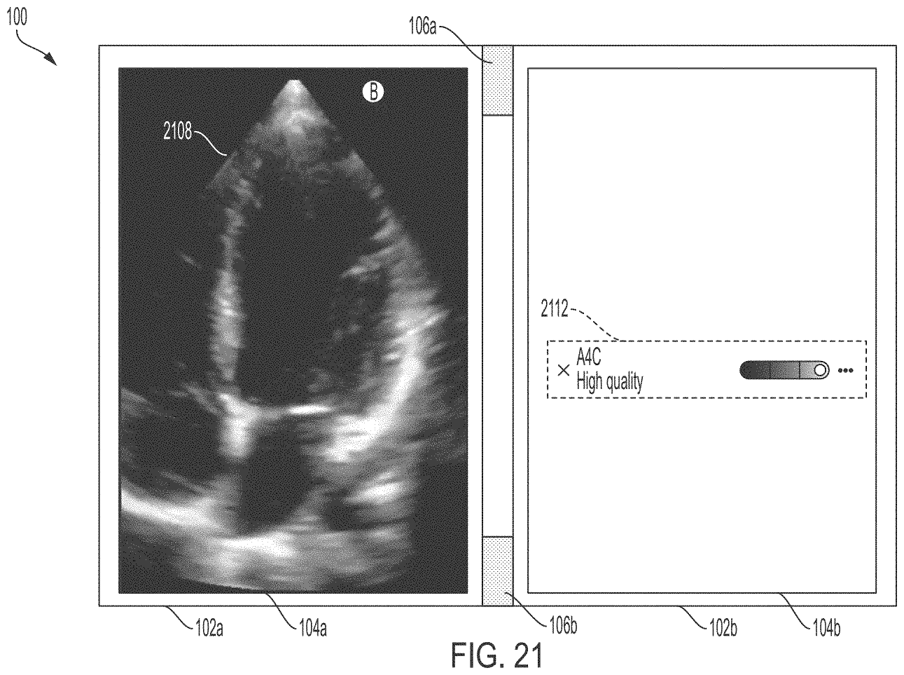

FIG. 21 illustrates the foldable processing device 100 when imaging the heart, in accordance with certain embodiments described herein. The first display screen 104a displays an ultrasound image 2108 and the second display screen 104b displays a quality indicator 2112 indicating a quality of the ultrasound image 2108. The ultrasound image 2108 may be the most recently displayed ultrasound image, and may be frozen on the display screen 104a or updated in real time as subsequent ultrasound images are collected. In some embodiments, the quality of the ultrasound image 2108 as indicated by the quality indicator 2112 may be based, at least in part, on a prediction of what proportion of experts (e.g., experts in the field of medicine, experts in a particular field of medicine, experts in ultrasound imaging, etc.) would consider the ultrasound image 2108 clinically usable as an ultrasound image of the heart. In some embodiments, to determine the quality as indicated by the quality indicator 2112, the foldable processing device 100 may use a statistical model trained to output such a prediction based on inputted ultrasound images. The quality indicator 2112 may be specific to ultrasound imaging of the heart in that it may indicate a low quality for ultrasound images of other anatomical portions despite such ultrasound images being high quality otherwise. This may be due to the statistical model being specifically trained to recognize ultrasound images of the heart as high quality. The quality indicator 2112 may specifically indicate high qualities for ultrasound images predicted to be usable for certain purposes related to ultrasound imaging of the heart, such as for calculating ejection fraction. Further description of determining and the quality of an ultrasound image may be found in U.S. patent application Ser. No. 16/880,272 titled “METHODS AND APPARATUSES FOR ANALYZING IMAGING DATA,” filed on May 21, 2020 (and assigned to the assignee of the instant application) and published as U.S. Pat. Pub. No. US 2020/0372657 A1, which is incorporated by reference herein in its entirety. As illustrated in FIG. 21, the quality indicator 2112 may indicate the quality textually, graphically, or both.

In some embodiments, the foldable processing device 100 may be configured to display the ultrasound image 2108 on the first display screen 104a and the quality indicator 2112 on the second display screen 104b based on receiving a selection from a user (e.g., from a menu of options displayed on either or both of the first display screen 104a and the second display screen 104b) to image the heart. Such selection may cause the foldable processing device 100 to configure the ultrasound device 124 with predetermined imaging parameters (which may be referred to as a preset) optimized for imaging the heart. In some embodiments, the foldable processing device 100 may be configured to display the ultrasound image 2108 on the first display screen 104a and the quality indicator 2112 on the second display screen 104b based on an automatic selection by the foldable processing device 100 (e.g., as part of an automatic workflow) to image the heart.

While the above description has focused on a quality indicator for ultrasound images of the heart, it should be appreciated that this application is not limited to the heart, and the foldable processing device 100 may display quality indicators actions related to ultrasound imaging of other anatomical portions. For example, the foldable processing device 100 may display quality indicators indicating how clinically usable an ultrasound image is as an ultrasound image of the lungs, as an ultrasound image of the bladder, or as an ultrasound image of a fetus. Such quality indicators may specifically indicate high qualities for ultrasound images predicted to be usable for certain purposes related to ultrasound imaging of other anatomical portions, such as for counting B-lines in lung imaging, for calculating bladder volume in bladder imaging, or for calculating gestational age, estimated delivery date, fetal weight, or amniotic fluid index in obstetric imaging.

While FIG. 21 illustrates the ultrasound image 2108 on the first display screen 104a, in some embodiments the ultrasound image 2108 may be on the second display screen 104b. While FIG. 21 illustrates the quality indicator 2112 on the second display screen 104b, in some embodiments the subject image quality indicator 2112 may be on the first display screen 104a.

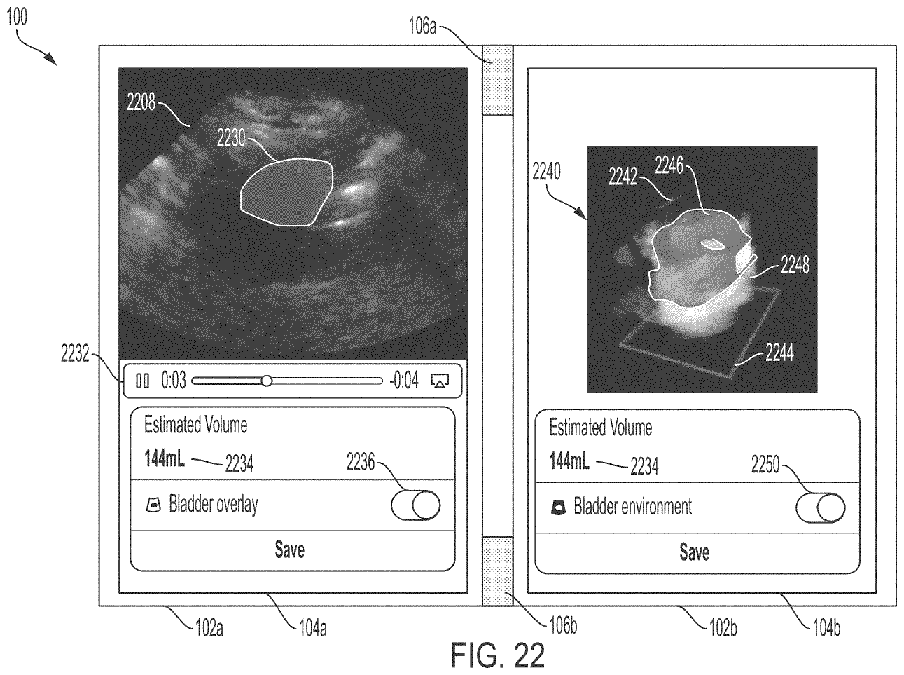

FIG. 22 illustrates the foldable processing device 100 when imaging the bladder, in accordance with certain embodiments described herein. The foldable processing device 100 may display imaging results of a 3D imaging sweep a bladder. The 3D sweep may be an elevational sweep. In other words, during the 3D sweep, the ultrasound device 124 may collect multiple ultrasound images, each ultrasound image collected along a different imaging slice at a different angle along the elevational dimension of the transducer array of the ultrasound device 124. The ultrasound device 124 may use beamforming to focus an ultrasound beam along a different direction at each stage of the 3D sweep. The 3D sweep may be performed while the user maintains the ultrasound device 124 at a constant position and orientation. The ultrasound device 124 may use a two-dimensional array of ultrasound transducers on a chip to perform the three-dimensional ultrasound imaging sweep while the user maintains the ultrasound device at a constant position and orientation. The beamforming process may include applying different delays to the transmitted and received ultrasound waves/data from different portions of the ultrasound transducer array (e.g., different delays for different elevational rows, where a row refer to a sequence of elements at the same position on the short axis of the ultrasound transducer array).

The first display screen 104a displays 2D imaging results of the 3D imaging sweep. In particular, the first display screen 104a displays an ultrasound image 2208 that is a part of a cine, a segmented portion 2230, a cine control/information bar 2232, a measurement value indicator 2234, and a bladder overlay option 2236. The cine may display the ultrasound images collected during the 3D imaging sweep, one after another. For example, the cine may first display the ultrasound image collected at the first elevational angle used during the 3D imaging sweep, then display the ultrasound image collected at the second elevational angle used during the 3D imaging sweep, etc. In FIG. 22, one ultrasound image 2208 of the cine is displayed on the first display screen 104a, but it should be appreciated that after a period of time the first display screen 104a may next display a next ultrasound image in the cine.

The cine control/information bar 2232 may control and provide information about the cine. For example, the cine control/information bar 2232 may provide information about how much time has elapsed during playback of the cine, how much time remains for playback of the cine, and may control playing, pausing, or changing to a different point in the cine. In some embodiments, the cine may play in a loop.

The segmented portion 2230 may represent the interior of the bladder as depicted in the ultrasound image 2208. In some embodiments, the foldable processing device 100 may use a statistical model to generate the segmented portion 2230. In particular, the statistical model may be trained to determine the location for segmented portions in ultrasound images. The bladder overlay option 2236 may toggle display of such segmented portions on or off.

The measurement value indicator 2234 may display a value for a measurement performed on the ultrasound images collected during the sweep. For example, the measurement may be a measurement of the volume of the bladder depicted in the ultrasound images collected during the sweep. In some embodiments, to perform a volume measurement, the foldable processing device 100 may calculate the area of the segmented portions (if any) in each ultrasound image collected during the sweep. The processing device may then calculate the average area of the segmented portions in each successive pair of ultrasound images in the 3D sweep (e.g., the average of the segmented portions in the first and second ultrasound images, the average of the segmented portions in second and third ultrasound images, etc.). The processing device may then multiply each averaged area by the angle (in radians) between each successive imaging slice in the 3D sweep to produce a volume, and sum all the volumes to produce the final volume value. It should be appreciated that other methods for performing measurements based on ultrasound images may be used, and other types of measurements may also be performed.

The second display screen 104b displays a 3D visualization 2240 that includes a first orientation indicator 2242, and a second orientation indicator 2244, a 3D bladder visualization 2246, and a 3D environment visualization 2248. The second display screen 104b further includes a bladder environment option 2250 and the measurement value indicator 2234. The 3D visualization 2140 may be generated from the ultrasound images collected during the 3D sweep and segmented portions from the ultrasound images. The 3D bladder visualization 2246 may depict the 3D volume of the bladder and the 3D environment visualization 2248 may depict surrounding tissue in 3D. The bladder environment option 2250 may toggle display of the 3D environment visualization 2248 on or off. Thus, if the bladder environment option 2250 is set on, the 3D bladder visualization 2246 and the 3D environment visualization 2248 may be displayed, and if the bladder environment option 2250 is set off, the 3D bladder visualization 2246 but not the 3D environment visualization 2248 may be displayed.

In some embodiments, the first orientation indicator 2242 may be an indicator of the position of the ultrasound device that performed the 3D sweep relative to the bladder depicted by the 3D visualization 2240. In some embodiments, the second orientation indicator 2244 may be an indicator of the position of the bottom plane of the ultrasound images collected during the 3D sweep relative to the bladder depicted by the 3D visualization 2240. Thus, the positions of the first orientation indicator 2242 and/or the second orientation indicator 2244 relative to the 3D visualization 2240 may provide information about the orientation of the 3D visualization 2240 as depicted on the second display screen 104b.

In some embodiments, the foldable processing device 100 may detect a dragging or pinching movement across its touch-sensitive second display screen 104b and, based on the dragging or pinching movement, modify the display of the 3D visualization 2240, the first orientation indicator 2242, and the second orientation indicator 2244 to depict them as if they were being rotated and/or zoomed in three dimensions. For example, in response to a horizontal dragging movement across the second display screen 104b of the foldable processing device 100, the foldable processing device 100 may display the 3D visualization 2240, the first orientation indicator 2242, and the second orientation indicator 2244 such that they appear to be rotated in three dimensions about a vertical axis. In response to a vertical dragging movement, foldable processing device 100 may display the 3D visualization 2240, the first orientation indicator 2242, and the second orientation indicator 2244 such that they appear to be rotated in three dimensions about a horizontal axis. In response to a pinching movement, foldable processing device 100 may display the 3D visualization 2240, the first orientation indicator 2242, and the second orientation indicator 2244 such that they appear zoomed in.

The foldable processing device 100 may advantageously allow a user to view 2D bladder images on the first display screen 104a and a 3D bladder visualization on the second display screen 104b simultaneously. Further description of 3D sweeps, generating segmented portions, displaying cines, generating 3D visualizations, and other aspects of bladder imaging may be found in U.S. Patent Publication No. 2020/0320694 A1 titled “METHODS AND APPARATUSES FOR COLLECTION AND VISUALIZATION OF ULTRASOUND DATA,” published on Oct. 8, 2020 (and assigned to the assignee of the instant application), which is incorporated by reference herein in its entirety.

While FIG. 22 illustrates the 2D ultrasound image 2208 on the first display screen 104a, in some embodiments the 2D ultrasound image 2208 may be on the second display screen 104b. While FIG. 22 illustrates the 3D visualization 2240 on the second display screen 104b, in some embodiments the 3D visualization 2240 may be on the first display screen 104a. While FIG. 22 and the associated description illustrate and describe 3D imaging sweeps of a bladder, 3D imaging sweeps of other anatomies may be used, and the foldable processing device 100 may display 2D images and 3D visualizations of these other anatomies in the same manner as described above for a bladder.

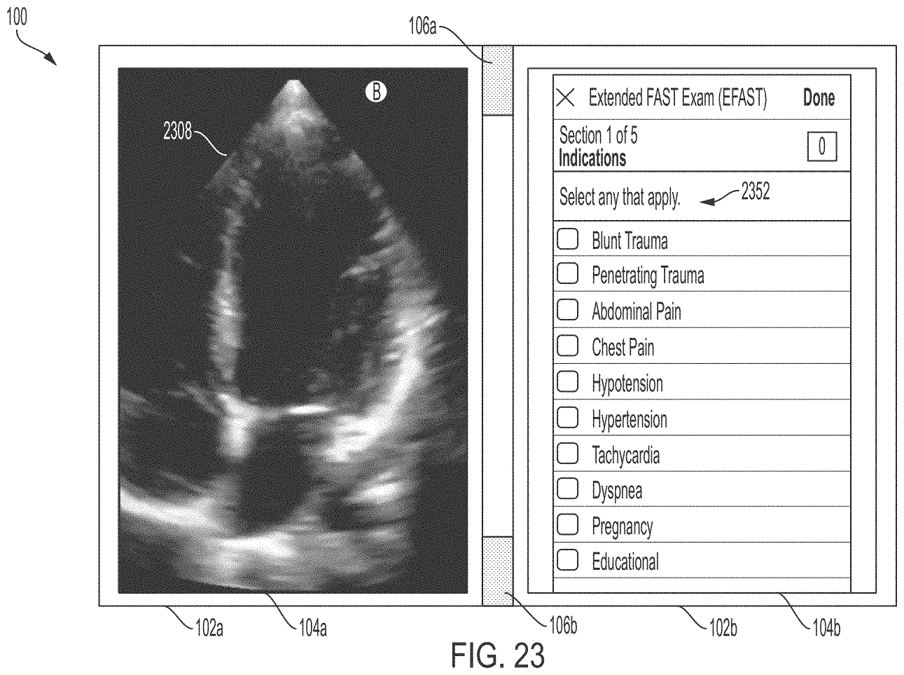

FIG. 23 illustrates the foldable processing device 100 when performing ultrasound imaging and documentation, in accordance with certain embodiments described herein. The first display screen 104a displays an ultrasound image 2308, which may be frozen on the first display screen 104a or updated in real time with new ultrasound images. The second display screen 104b displays fillable documentation 2352. A user may fill out the fillable documentation 2352, and may use the ultrasound image 2308 as a reference when doing so. The fillable documentation 2352 may include, for example, documentation for indications, views, findings, interpretation, and Current Procedural Terminology (CPT) codes. The fillable documentation 2352 may include, for example, dropdown fields, radio buttons, checkboxes, and/or text fields for which a user may provide selections and/or inputs. The user may advantageously view one or more ultrasound images 2352 on the first display screen 104a while simultaneously completing the fillable documentation 2352 on the second display screen 104b. The foldable processing device 100 may store the user selections and/or inputs on the foldable processing device 100 and/or on a remote server. The foldable processing device 100 may associate the user selections and/or inputs with the ultrasound image 2308 and/or an imaging study of which the ultrasound image 2308 is a part.

While FIG. 23 illustrates the ultrasound image 2308 on the first display screen 104a, in some embodiments the ultrasound image 2308 may be on the second display screen 104b. While FIG. 23 illustrates the fillable documentation 2352 on the second display screen 104b, in some embodiments the fillable documentation 2352 may be on the first display screen 104a.

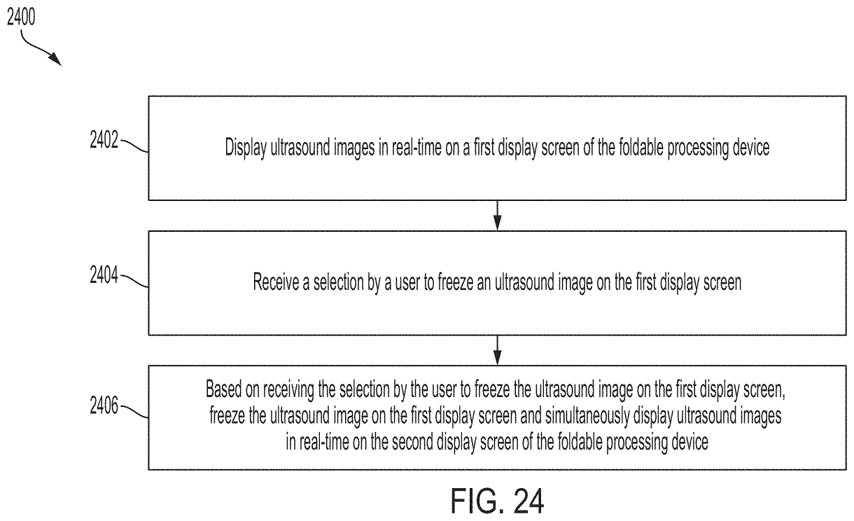

FIG. 24 illustrates a process 2400 for using the foldable processing device 100 to view ultrasound images in real-time and to freeze ultrasound images on a display screen, in accordance with certain embodiments described herein.

In act 2402, the foldable processing device 100 displays ultrasound images in real-time on the first display screen 104a of the foldable processing device 100. Thus, during the process 2400, the ultrasound device 124 may be collecting ultrasound data in real-time, and as new ultrasound data is collected, the first display screen 104a may replace the ultrasound image displayed on the first display screen 104a with a new ultrasound image generated based on the ultrasound data most recently collected by the ultrasound device 124. In some embodiments, during act 2402, ultrasound images in real-time may not be displayed on the second display screen 104b. The process 2400 proceeds from act 2402 to act 2404.

In act 2404, the foldable processing device 100 receives a selection by a user to freeze an ultrasound image on the first display screen 104a. The ultrasound image may be one of the ultrasound images displayed in real-time in act 2402. The foldable processing device 100 may receive the selection through controls displayed on the first display screen 104a and/or on the second display screen 104b (e.g., the ultrasound imaging controls 1714). The user may select the controls by touching the display screen, for example. The process 2400 proceeds from act 2404 to act 2406.

In act 2406, based on receiving the selection by the user to freeze the ultrasound image on the first display screen 104a in act 2404, the foldable processing device 100 freezes the ultrasound image on the first display screen 104a and simultaneously displays ultrasound images in real-time on the second display screen 104b of the foldable processing device 100. The foldable processing device 100 may display the ultrasound images in real-time on the second display screen 104b in the same manner that it displayed the ultrasound images in real-time on the first display screen 104a in act 2402. The user may also cause an ultrasound image to freeze on the second display screen 104b in the same manner as described above with reference to the first display screen 104a in act 2404. Thus, the user may advantageously view the frozen ultrasound image on the first display screen 104a and the real-time ultrasound images and/or frozen ultrasound image on the second display screen 104b simultaneously.

In some embodiments, at act 2402, the foldable processing device 100 may display ultrasound images in real-time on the second display screen 104b. At act 2404, the foldable processing device 100 may receive a selection by a user to freeze an ultrasound image on the second display screen 104b. At act 2406, based on receiving the selection by the user to freeze the ultrasound image on the second display screen 104b, the foldable processing device 100 may freeze the ultrasound image on the second display screen 104a and display ultrasound images in real-time on the first display screen 104a of the foldable processing device 100.

It should be appreciated that any of the items described and/or illustrated above as displayed on the first display screen 104a or the second display screen 104b of the foldable processing device 100 may be displayed together. For example, any combination of ultrasound images (e.g., the ultrasound image the azimuthal plane 408, the ultrasound image along the elevational plane 410, or the ultrasound images 608, 808, 1208, 1708, 1808, 1908, 2108, 2308), ultrasound image displayed as a cine (e.g., the ultrasound image 2208), velocity trace (e.g., the velocity trace 610), M-mode trace (e.g., the M-mode trace 810), actions (e.g., the actions related to ultrasound imaging of the heart 1210), quality indicators (e.g., the quality indicator 2112), ultrasound imaging controls (e.g., the ultrasound imaging controls 1714), subject images (e.g., the subject image 1816), remote guide images (e.g., the remote guide image 1818), telemedicine controls (e.g., the telemedicine controls 1820), set of previously-collected ultrasound images 1922, 3D visualization (e.g., the 3D visualization 2240), and/or fillable documentation 2352 may be displayed together on the same display screen (e.g., either on the first display screen 104a or the second display screen 104b).

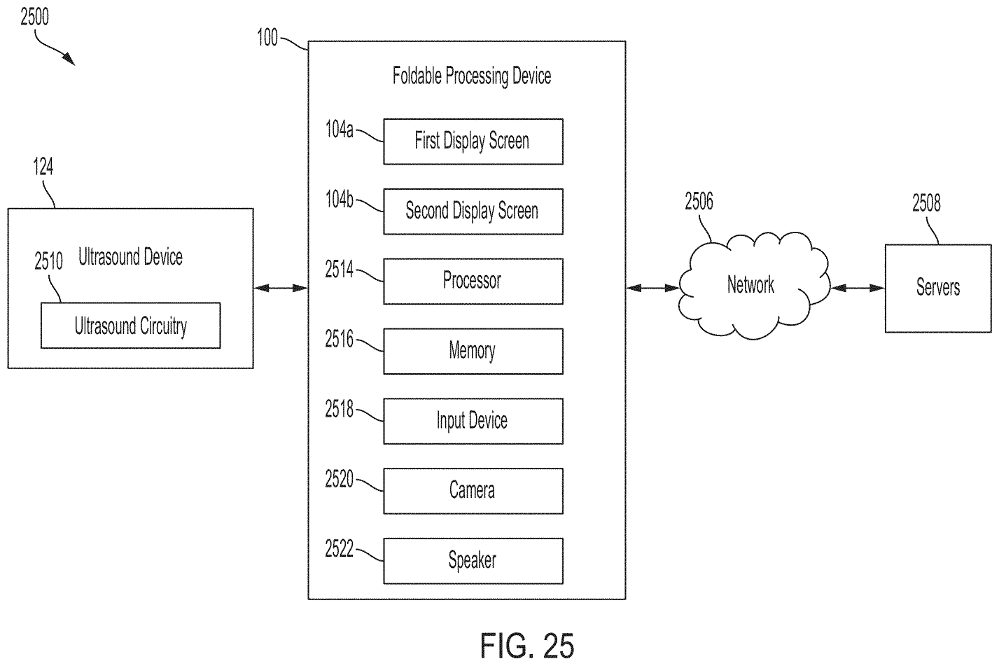

FIG. 25 illustrates a schematic block diagram of an example ultrasound system 2500 upon which various aspects of the technology described herein may be practiced. The ultrasound system 2500 includes an ultrasound device 124, the foldable processing device 100, a network 2506, and one or more servers 2508.

The ultrasound device 124 includes ultrasound circuitry 2510. The foldable processing device 100 includes a camera 2520, the first display screen 104a, the second display screen 104b, a processor 2514, a memory 2516, an input device 2518, a camera 2520, and a speaker 2522. The foldable processing device 100 is in wired (e.g., through an Ethernet cable, a Universal Serial Bus (USB) cable, or a Lightning cable,) and/or wireless communication (e.g., using BLUETOOTH, ZIGBEE, and/or WiFi wireless protocols) with the ultrasound device 124. The illustrated communication link between the ultrasound device 124 and the foldable processing device 100 may be the cable 126 shown in FIG. 1. The foldable processing device 100 is in wireless communication with the one or more servers 2508 over the network 2506.

The ultrasound device 124 may be configured to generate ultrasound data that may be employed to generate an ultrasound image. The ultrasound device 124 may be constructed in any of a variety of ways. In some embodiments, the ultrasound device 124 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 2510 may be configured to generate the ultrasound data. The ultrasound circuitry 2510 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 2510 (e.g., transmit circuitry, receive circuitry, control circuitry, power management circuitry, and processing circuitry) to form a monolithic ultrasound device. The ultrasound device 124 may transmit ultrasound data and/or ultrasound images to the foldable processing device 100 over a wired (e.g., through an Ethernet cable, a Universal Serial Bus (USB) cable, or a Lightning cable,) and/or wireless (e.g., using BLUETOOTH, ZIGBEE, and/or WiFi wireless protocols) communication link. The wired communication link may include the cable 126.

Referring now to the foldable processing device 100, the processor 2514 may include specially-programmed and/or special-purpose hardware such as an application-specific integrated circuit (ASIC). For example, the processor 2514 may include one or more graphics processing units (GPUs) and/or one or more tensor processing units (TPUs). TPUs may be ASICs specifically designed for machine learning (e.g., deep learning). The TPUs may be employed, for example, to accelerate the inference phase of a neural network. The foldable processing device 100 may be configured to process the ultrasound data received from the ultrasound device 124 to generate ultrasound images or other types of displays related to particular ultrasound imaging modes (e.g., velocity traces or M-mode traces) for display on the first display screen 104a and/or the second display screen 104b. The processing may be performed by, for example, the processor 2514. The processor 2514 may also be adapted to control the acquisition of ultrasound data with the ultrasound device 124. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional images generated from more-recently acquired ultrasound data may be sequentially displayed (and, in certain ultrasound image modes, various other types of displays such as velocity traces or M-mode traces may be updated based on the newly acquired ultrasound images). Additionally, or alternatively, the ultrasound data may be stored temporarily in a buffer during a scanning session and processed in less than real-time.

The foldable processing device 100 may be configured to perform certain of the processes (e.g., the processes 1000, 1100, 1300, 1400, 1500, 1600, 2000, and/or 2400) described herein using the processor 2514 (e.g., one or more computer hardware processors) and one or more articles of manufacture that include non-transitory computer-readable storage media such as the memory 2516. The processor 2514 may control writing data to and reading data from the memory 2516 in any suitable manner. To perform certain of the processes described herein (e.g., the processes 1000, 1100, 1300, 1400, 1500, 1600, 2000, and/or 2400), the processor 2514 may execute one or more processor-executable instructions stored in one or more non-transitory computer-readable storage media (e.g., the memory 2516), which may serve as non-transitory computer-readable storage media storing processor-executable instructions for execution by the processor 2514. The camera 2520 may be configured to detect light (e.g., visible light) to form an image. The camera 2520 may be on the same face of the foldable processing device 100 as the first display screen 104a or the second display screen 104b. The first display screen 104a and the second display screen 104b may be configured to display images and/or videos, and may each be, for example, a liquid crystal display (LCD), a plasma display, and/or an organic light emitting diode (OLED) display on the foldable processing device 100. The input device 2518 may include one or more devices capable of receiving input from a user and transmitting the input to the processor 2514. For example, the input device 2518 may include a keyboard, a mouse, a microphone, touch-enabled sensors on the first display screen 104a and/or the second display screen 104b, and/or a microphone. The first display screen 104a, the second display screen 104b, the input device 2518, the camera 2520, and the speaker 2522 may be communicatively coupled to the processor 2514 and/or under the control of the processor 2514.

It should be appreciated that the foldable processing device 100 may be implemented in any of a variety of ways. For example, the foldable processing device 100 may be implemented as a handheld device such as a mobile smartphone or a tablet. Thereby, a user of the ultrasound device 124 may be able to operate the ultrasound device 124 with one hand and hold the foldable processing device 100 with another hand. In other examples, the foldable processing device 100 may be implemented as a portable device that is not a handheld device, such as a laptop. In yet other examples, the foldable processing device 100 may be implemented as a stationary device such as a desktop computer. The foldable processing device 100 may be connected to the network 2506 over a wired connection (e.g., via an Ethernet cable) and/or a wireless connection (e.g., over a WiFi network). The foldable processing device 100 may thereby communicate with (e.g., transmit data to or receive data from) the one or more servers 2508 over the network 2506. For example, a party may provide from the server 2508 to the foldable processing device 100 processor-executable instructions for storing in one or more non-transitory computer-readable storage media (e.g., the memory 2516) which, when executed, may cause the foldable processing device 100 to perform certain of the processes (e.g., the processes 1000, 1100, 1300, 1400, 1500, 1600, 2000, and/or 2400) described herein.

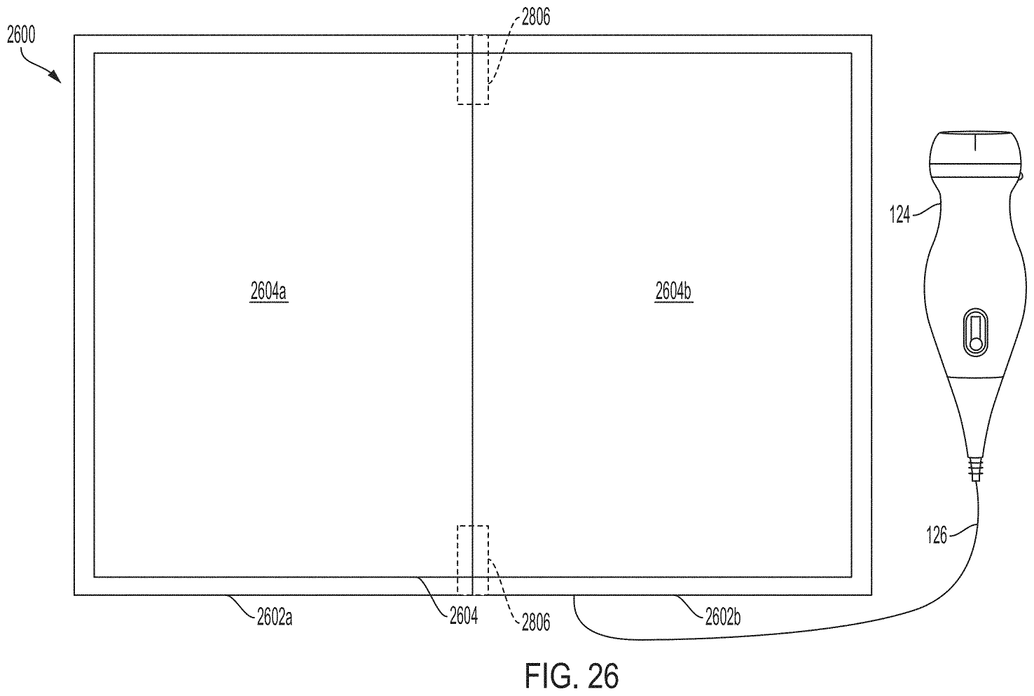

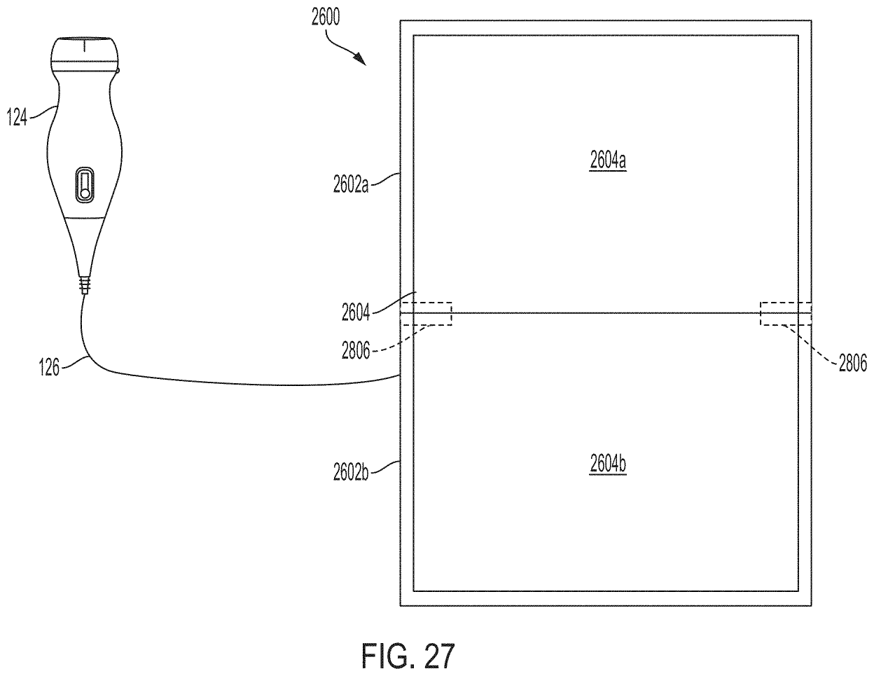

FIG. 26 illustrates a top view of a foldable processing device 2600 in an open configuration, in accordance with certain embodiments described herein. The foldable processing device 2600 may be any type of processing device, such as a mobile smartphone or a tablet. The foldable processing device 2600 includes a first panel 2602a, a second panel 2602b, and a display screen 2604. The first panel 2602a and the second panel 2602b are rotatably coupled by a hinge 2806, shown in dashed lines in FIGS. 26 and 27 because it is obstructed by the display screen 2604 in the views of those two figures. The display screen 2604 extends from the first panel 2602a to the second panel 2602b. In some embodiments, the display screen 2604 extends through the hinge 2806. In some embodiments, the display screen 2604 passes in front of the hinge 2806. That is, in some embodiments the hinge 2806 is positioned behind the display screen 2604. While the display screen 2604 is a single, unitary display screen, it may be considered to have two portions, a first display screen portion 2604a and a second display screen portion 2604b, each representing half of the display screen portion 2604 on either side of the hinge 2806. While the display screen 2604 may display a single display, in some embodiments, as will be described further below, the first display screen portion 2604a may display one display and the second display screen portion 2604b may depict a different display. FIG. 26 further illustrates the ultrasound device 124 and the cable 126.

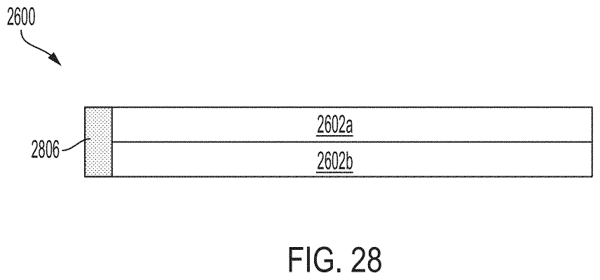

FIG. 26 displays an open configuration for the foldable processing device 2600 in which the first panel 2602a and the second panel 2602b are substantially coplanar, and the display screen 2604 is visible to a user. The hinge 2806 enables the first panel 2602a and/or the second panel 2602b to rotate about the hinge 2806 such that the foldable processing device 2600 goes from the open configuration to a folded configuration, as illustrated in the side view of FIG. 28.

FIG. 27 illustrates another top view of the foldable processing device 2600 in the open configuration, in accordance with certain embodiments described herein. The foldable processing device 2600 is illustrated rotated from the orientation in FIG. 26. In some embodiments, in response to rotation of the foldable processing device 2600 from the orientation in FIG. 26 to the orientation in FIG. 27, or vice versa, the foldable processing device 2600 may cause the displays that are displayed on the first display screen portion 2604a and/or the second display screen portion 2604b to rotate as well. The configuration of FIG. 26 may be referred to as portrait mode while the configuration of FIG. 27 may be referred to as landscape mode.

FIG. 28 illustrates a side view of the foldable processing device 2600 in a folded configuration, in accordance with certain embodiments described herein. In the folded configuration, the display screen 2604 may fold upon itself, such that the first display screen portion 2604a and the second display screen portion 2604b face each other, may be in contact with each other, and may not be visible to a user. The first panel 2602a and the second panel 2602b may be stacked one on top of another. The hinge 2806 enables the first panel 2602a and/or the second panel 2602b to rotate about the hinge 2806 such that the foldable processing device 2600 goes from the folded configuration to the open configuration, as illustrated in FIGS. 26 and 27. As described above, the display screen may extend from the first panel 2602a, through or in front of the hinge 2806, and to the second panel 2602b, such that the display screen 2604 is a single display screen that can fold upon itself along the hinge 2806. Thus, the display screen 2604 may be considered to be foldable. The foldable processing device 2600 may be more compact in the folded configuration than in the open configuration, while the open configuration may allow the display screen 2604 to be visible. The display screen 2604, by virtue of being foldable, may provide a relatively large display screen when the foldable processing device 2600 is opened while providing a relatively small form factor when the foldable processing device 2600 is folded.

While FIGS. 26-28 illustrate two hinges 2806, some embodiments may have one or more hinges, and the hinges may be at different locations. Additionally, other means for coupling the first panel 2602a and the second panel 2602b together such that the foldable processing device 2600 can go from an open configuration to a folded configuration may be used. For example, the foldable processing device 2600 may be formed of a foldable sheet of continuous material, such as a flexible circuit. It should also be appreciated that the size and shape of the foldable processing device 2600, the first panel 2602a, the second panel 2602b, and the display screen 2604 as illustrated is non-limiting, and that the foldable processing device 2600, the first panel 2602a, the second panel 2602b, and the display screen 2604 may have different sizes and/or shapes than illustrated.

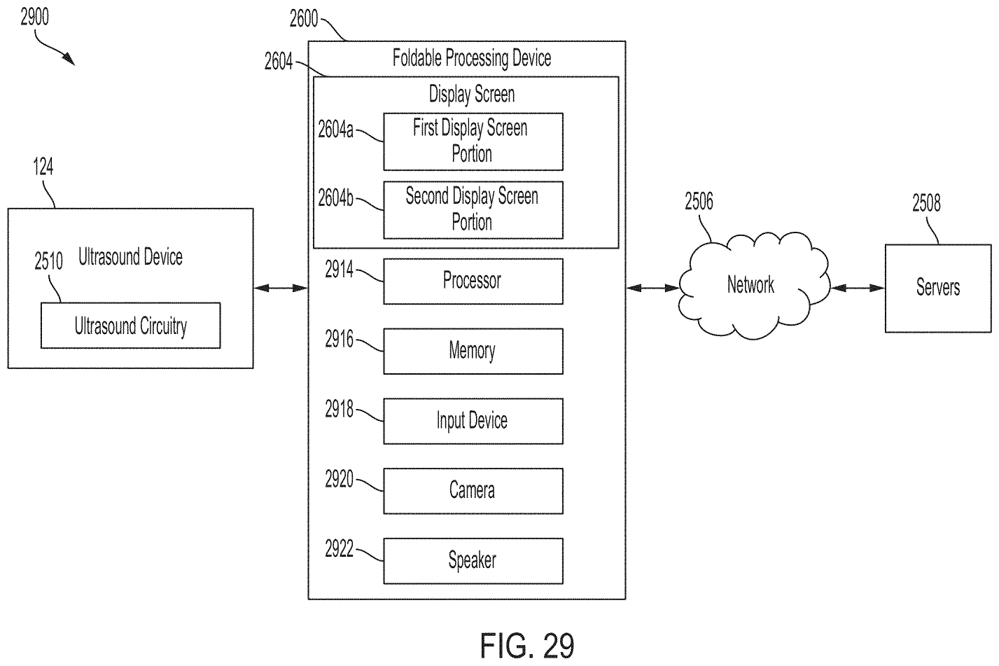

FIG. 29 illustrates a schematic block diagram of an example ultrasound system 2900 upon which various aspects of the technology described herein may be practiced. The ultrasound system 2900 includes the ultrasound device 124, the foldable processing device 2600, the network 2506, and the one or more servers 2508.

The foldable processing device 2600 includes the display screen 2604, a processor 2914, a memory 2916, an input device 2918, a camera 2920, and a speaker 2922. The display screen 2604 has a first display portion 2604a and a second display portion 2604b. Further description of the foldable processing device 2600, the display screen 2604, the processor 2914, the memory 2916, the input device 2918, the camera 2920, and the speaker 2922 may be found with reference to the foldable processing device 100, the first display screen 104a and the second display screen 104b, the processor 2514, the memory 2516, the input device 2518, the camera 2520, and the speaker 2522 described above.

Any of the features and operation of the foldable processing device 100, the first display screen 104a, and the second display screen 104b described above may also be implemented in the foldable processing device 2600, the first display screen portion 2604a of the display screen 2604, and the second display screen portion 2604b of the display screen 2604, respectively. In other words, for any application in which a first display is described above as displayed on the first display screen 104a of the foldable processing device 100 and a second display is described above as displayed on the second display screen 104b of the foldable processing device 100, the first display may instead be displayed on the first display screen portion 2604a of the foldable processing device 2600 and the second display may instead be displayed on the second display screen portion 2604b of the foldable processing device 2600. Thus, in any of FIGS. 4-9, 12, 17-19, and 21-23, the display shown on the first display screen 104a of the foldable processing device 100 may be shown on the first display screen portion 2604a of the foldable processing device 2600, and the display shown on the second display screen 104b of the foldable processing device 100 may be shown on the second display screen portion 2604b. In any of processes 1000, 1100, 1300, 1400, 1500, 1600, 2000, and/or 2400, the display shown on the first display screen 104a of the foldable processing device 100 may be shown on the first display screen portion 2604a of the foldable processing device 2600, and the display shown on the second display screen 104b of the foldable processing device 100 may be shown on the second display screen portion 2604b. As a particular example, the first display screen portion 2604a may display an ultrasound image along the elevational plane and the second display screen portion 2604b may display an ultrasound image along the azimuthal plane, corresponding to the configuration of FIG. 4.

In a first group of embodiments, a foldable processing device is provided, comprising: a first panel; a second panel; one or more hinges, wherein the first panel and the second panel are rotatably coupled by the one or more hinges; and a foldable display screen extending between the first panel and the second panel, configured to fold upon itself about the one or more hinges, and comprising a first display screen portion and a second display screen portion, each on a different side of the one or more hinges. The foldable processing device is in operative communication with an ultrasound device. In a second group of embodiments, a foldable processing device is provided, comprising: a first panel comprising a first display screen; a second panel comprising a second display screen; and one or more hinges, wherein the first panel and the second panel are rotatably coupled by the one or more hinges. In any of the first and second groups of embodiments, the foldable processing device may be in operative communication with an ultrasound device.

In any of the first and second groups of embodiments of a foldable processing device, the foldable processing device may be configured to simultaneously: display an ultrasound image along an elevational plane on the first display screen or display screen portion; and display an ultrasound image along an azimuthal plane on the second display screen or display screen portion.

In any of the first and second groups of embodiments of a foldable processing device, the foldable processing device may be configured to simultaneously: display an ultrasound image on the first display screen or display screen portion; and display a pulsed wave Doppler imaging mode velocity trace on the second display screen or display screen portion.

In any of the first and second groups of embodiments of a foldable processing device, the foldable processing device may be configured to simultaneously: display an ultrasound image on the first display screen or display screen portion; and display an M-mode trace on the second display screen or display screen portion.

In any of the first and second groups of embodiments of a foldable processing device, the foldable processing device may be configured to simultaneously: display an ultrasound image on the first display screen or display screen portion; and display actions related to ultrasound imaging of an anatomical portion on the second display screen or display screen portion. The actions related to ultrasound imaging of the anatomical portion comprise actions performed by the foldable processing device that enable a user: to annotate the ultrasound image with annotations specific to the anatomical portion; to be guided by the foldable processing device to collect an ultrasound image of the anatomical portion; to cause the foldable processing device to automatically perform a calculation related to the anatomical portion, wherein the calculation related to the anatomical portion comprises calculation of ejection fraction, counting of B-lines, calculation of bladder volume, calculation of gestational age, calculation of estimated delivery date, calculation of fetal weight, and/or calculation of amniotic fluid index; and/or to view a video related to ultrasound imaging of the anatomical portion.

In any of the first and second groups of embodiments of a foldable processing device, the foldable processing device may be configured to simultaneously: display an ultrasound image on the first display screen or display screen portion; and display a quality indicator for the ultrasound image related to ultrasound imaging of an anatomical portion on the second display screen or display screen portion.

In any of the first and second groups of embodiments of a foldable processing device, the foldable processing device may be configured to: display an ultrasound image on the first display screen or display screen portion; and display ultrasound imaging controls on the second display screen or display screen portion, wherein the ultrasound imaging controls comprise controls for freezing the ultrasound image, capturing the ultrasound image as a still image, recording an ultrasound clip, adjusting gain, adjusting depth, adjusting time gain compensation (TGC), selecting an anatomical portion to be imaged, selecting an ultrasound imaging mode, annotating the ultrasound image, and/or performing measurements on the ultrasound image.

In any of the first and second groups of embodiments of a foldable processing device, the foldable processing device may be configured to: display an ultrasound image on the first display screen or display screen portion; and display a portion of a telemedicine interface on the second display screen or display screen portion, wherein: the telemedicine interface comprises a subject image, a remote guide image, and/or telemedicine controls; the subject image is a frame of a video captured by a camera of the foldable processing device and shows a subject being imaged, the ultrasound device, and an instruction for moving the ultrasound device; and the instruction comprises an instruction to translate, rotate, or tilt the ultrasound device.

In any of the first and second groups of embodiments of a foldable processing device, the foldable processing device may be configured to: display a set of saved ultrasound images on the second display screen or display screen portion as thumbnails; receive a selection by a user of an ultrasound image or image(s) from the set of saved ultrasound images; and display the ultrasound image or image(s) on the first display screen or display screen portion at a larger size than they are displayed on the second display screen or display screen portion.

In any of the first and second groups of embodiments of a foldable processing device, the foldable processing device may be configured to: display an ultrasound image on the first display screen or display screen portion; display fillable documentation on the second display screen or display screen portion, wherein the fillable documentation comprises a dropdown field, radio button, checkbox, and text field for which a user may provide selection and/or input; and store the user selection and/or input on the foldable processing device and/or on a remote server.

In any of the first and second groups of embodiments of a foldable processing device, the foldable processing device may be configured to: display an ultrasound image of a bladder on the first display screen or display screen portion; and display a three-dimensional visualization of the bladder on the second display screen or display screen portion.

In any of the first and second groups of embodiments of a foldable processing device, the foldable processing device may be configured to: display ultrasound images in real-time on a first display screen or display screen portion of the foldable processing device; receive a selection by a user to freeze an ultrasound image on the first display screen or display screen portion; and based on receiving the selection by the user to freeze the ultrasound image on the first display screen or display screen portion, freeze the ultrasound image on the first display screen or display screen portion and simultaneously display ultrasound images in real-time on the second display screen or display screen portion of the foldable processing device.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A foldable processing device, comprising:
a first panel;
a second panel;
one or more hinges, wherein the first panel and the second panel are rotatably coupled by the one or more hinges; and
a foldable display screen extending between the first panel and the second panel, configured to fold upon itself about the one or more hinges, and comprising a first display screen portion and a second display screen portion, each on a different side of the one or more hinges;
wherein the foldable processing device is in operative communication with an ultrasound device.

2. The foldable processing device of claim 1, wherein the foldable processing device is configured to simultaneously:
display an ultrasound image along an elevational plane on the first display screen portion; and
display an ultrasound image along an azimuthal plane on the second display screen portion.

3. The foldable processing device of claim 1, wherein the foldable processing device is configured to simultaneously:
display an ultrasound image on the first display screen portion; and
display a pulsed wave Doppler imaging mode velocity trace on the second display screen portion.

4. The foldable processing device of claim 1, wherein the foldable processing device is configured to simultaneously:
display an ultrasound image on the first display screen portion; and
display an M-mode trace on the second display screen portion.

5. The foldable processing device of claim 1, wherein the foldable processing device is configured to simultaneously:
display an ultrasound image on the first display screen portion; and
display actions related to ultrasound imaging of an anatomical portion on the second display screen portion, wherein the actions related to ultrasound imaging of the anatomical portion comprise actions performed by the foldable processing device that enable a user:
to annotate the ultrasound image with annotations specific to the anatomical portion;
to be guided by the foldable processing device to collect an ultrasound image of the anatomical portion;
to cause the foldable processing device to automatically perform a calculation related to the anatomical portion, wherein the calculation related to the anatomical portion comprises calculation of ejection fraction, counting of B-lines, calculation of bladder volume, calculation of gestational age, calculation of estimated delivery date, calculation of fetal weight, and/or calculation of amniotic fluid index; and/or
to view a video related to ultrasound imaging of the anatomical portion.

6. The foldable processing device of claim 1, wherein the foldable processing device is configured to simultaneously:
display an ultrasound image on the first display screen portion; and
display a quality indicator for the ultrasound image related to ultrasound imaging of an anatomical portion on the second display screen portion.

7. The foldable processing device of claim 1, wherein the foldable processing device is configured to:
display an ultrasound image on the first display screen portion; and
display ultrasound imaging controls on the second display screen portion, wherein the ultrasound imaging controls comprise controls for freezing the ultrasound image, capturing the ultrasound image as a still image, recording an ultrasound clip, adjusting gain, adjusting depth, adjusting time gain compensation (TGC), selecting an anatomical portion to be imaged, selecting an ultrasound imaging mode, annotating the ultrasound image, and/or performing measurements on the ultrasound image.

8. The foldable processing device of claim 1, wherein the foldable processing device is configured to:
display an ultrasound image on the first display screen portion; and
display a portion of a telemedicine interface on the second display screen portion, wherein:
the telemedicine interface comprises a subject image, a remote guide image, and/or telemedicine controls;
the subject image is a frame of a video captured by a camera of the foldable processing device and shows a subject being imaged, the ultrasound device, and an instruction for moving the ultrasound device; and
the instruction comprises an instruction to translate, rotate, or tilt the ultrasound device.

9. The foldable processing device of claim 1, wherein the foldable processing device is configured to:
display a set of saved ultrasound images on the second display screen portion as thumbnails;
receive a selection by a user of an ultrasound image or image(s) from the set of saved ultrasound images; and
display the ultrasound image or image(s) on the first display screen portion at a larger size than they are displayed on the second display screen portion.

10. The foldable processing device of claim 1, wherein the foldable processing device is configured to:
display an ultrasound image on the first display screen portion;
display fillable documentation on the second display screen portion, wherein the fillable documentation comprises a dropdown field, radio button, checkbox, and text field for which a user may provide selection and/or input; and
store the user selection and/or input on the foldable processing device and/or on a remote server.

11. The foldable processing device of claim 1, wherein the foldable processing device is configured to:
display an ultrasound image of a bladder on the first display screen portion; and
display a three-dimensional visualization of the bladder on the second display screen portion.

12. The foldable processing device of claim 1, wherein the foldable processing device is configured to:
display ultrasound images in real-time on a first display screen portion of the foldable processing device;
receive a selection by a user to freeze an ultrasound image on the first display screen portion; and
based on receiving the selection by the user to freeze the ultrasound image on the first display screen portion, freeze the ultrasound image on the first display screen portion and simultaneously display ultrasound images in real-time on the second display screen portion of the foldable processing device.

13. A foldable processing device, comprising:
a first panel comprising a first display screen;
a second panel comprising a second display screen;
one or more hinges, wherein the first panel and the second panel are rotatably coupled by the one or more hinges; and
wherein the foldable processing device is in operative communication with an ultrasound device.

14. The foldable processing device of claim 13, wherein the foldable processing device is configured to simultaneously:
display an ultrasound image along an elevational plane on the first display screen; and
display an ultrasound image along an azimuthal plane on the second display screen.

15. The foldable processing device of claim 13, wherein the foldable processing device is configured to simultaneously:
display an ultrasound image on the first display screen; and
display a pulsed wave Doppler imaging mode velocity trace on the second display screen.

16. The foldable processing device of claim 13, wherein the foldable processing device is configured to simultaneously:
display an ultrasound image on the first display screen; and
display an M-mode trace on the second display screen.

17. The foldable processing device of claim 13, wherein the foldable processing device is configured to simultaneously:
display an ultrasound image on the first display screen; and
display actions related to ultrasound imaging of an anatomical portion on the second display screen, wherein the actions related to ultrasound imaging of the anatomical portion comprise actions performed by the foldable processing device that enable a user:
to annotate the ultrasound image with annotations specific to the anatomical portion;
to be guided by the foldable processing device to collect an ultrasound image of the anatomical portion;
to cause the foldable processing device to automatically perform a calculation related to the anatomical portion, wherein the calculation related to the anatomical portion comprises calculation of ejection fraction, counting of B-lines, calculation of bladder volume, calculation of gestational age, calculation of estimated delivery date, calculation of fetal weight, and/or calculation of amniotic fluid index; and/or
to view a video related to ultrasound imaging of the anatomical portion.

18. The foldable processing device of claim 13, wherein the foldable processing device is configured to simultaneously:
display an ultrasound image on the first display screen; and
display a quality indicator for the ultrasound image related to ultrasound imaging of an anatomical portion on the second display screen.

19. The foldable processing device of claim 13, wherein the foldable processing device is configured to:
display an ultrasound image on the first display screen; and
display ultrasound imaging controls on the second display screen, wherein the ultrasound imaging controls comprise controls for freezing the ultrasound image, capturing the ultrasound image as a still image, recording an ultrasound clip, adjusting gain, adjusting depth, adjusting time gain compensation (TGC), selecting an anatomical portion to be imaged, selecting an ultrasound imaging mode, annotating the ultrasound image, and/or performing measurements on the ultrasound image.

20. The foldable processing device of claim 13, wherein the foldable processing device is configured to:
display an ultrasound image on the first display screen; and
display a portion of a telemedicine interface on the second display screen, wherein:
the telemedicine interface comprises a subject image, a remote guide image, and/or telemedicine controls;
the subject image is a frame of a video captured by a camera of the foldable processing device and shows a subject being imaged, the ultrasound device, and an instruction for moving the ultrasound device; and
the instruction comprises an instruction to translate, rotate, or tilt the ultrasound device.

21. The foldable processing device of claim 13, wherein the foldable processing device is configured to:
display a set of saved ultrasound images on the second display screen as thumbnails;
receive a selection by a user of an ultrasound image or image(s) from the set of saved ultrasound images; and
display the ultrasound image or image(s) on the first display screen at a larger size than they are displayed on the second display screen.

22. The foldable processing device of claim 13, wherein the foldable processing device is configured to:
display an ultrasound image on the first display screen;
display fillable documentation on the second display screen, wherein the fillable documentation comprises a dropdown field, radio button, checkbox, and text field for which a user may provide selection and/or input; and
store the user selection and/or input on the foldable processing device and/or on a remote server.

23. The foldable processing device of claim 13, wherein the foldable processing device is configured to:
display an ultrasound image of a bladder on the first display screen; and
display a three-dimensional visualization of the bladder on the second display screen.

24. The foldable processing device of claim 13, wherein the foldable processing device is configured to:
display ultrasound images in real-time on a first display screen of the foldable processing device;
receive a selection by a user to freeze an ultrasound image on the first display screen; and
based on receiving the selection by the user to freeze the ultrasound image on the first display screen, freeze the ultrasound image on the first display screen and simultaneously display ultrasound images in real-time on the second display screen of the foldable processing device.
